(19) United States
(12) Patent Application Publication
(10) Pub. No.: US 2013/0095476 A1
(43) Pub. Date: Apr. 18, 2013

(54) DETECTION OF QUANTITATIVE GENETIC DIFFERENCES

(71) Applicant: Alexandre Loktionov, Cambridge (GB)

(72) Inventor: Alexandre Loktionov, Cambridge (GB)

(73) Assignee: ZORAGEN BIOTECHNOLOGIES LLP, London (GB)

(21) Appl. No.: 13/395,579

(22) PCT Filed: Sep. 9, 2010
(86) PCT No.: PCT/GB2010/051510

§ 371 Date: Nov. 28, 2012

(30) Foreign Application Priority Data
Sep. 12, 2009 (GB) 0916074.8
Sep. 12, 2009 (GB) 0916075.5

(51) Int. Cl. C12Q 1/68

(52) U.S. Cl. CPC C12Q 1/6851
USPC 435/6.11; 435/6.12

(57) ABSTRACT

A method for detection of a quantitative difference between the amount of a first target region of nucleic acid and a second target region of nucleic acid in a sample, comprising the steps of: providing the sample comprising the nucleic acid; amplifying the first and second target regions of the nucleic acid to obtain multiple copies of a first and a second sequence of nucleic acid; associating the amplified first sequence with the amplified second sequence to form associated nucleic acid complexes which comprise the first sequence and the second sequence in a 1:1 ratio, wherein any excess of either the first sequence or the second sequence remain un-associated; detecting any un-associated sequences, wherein detection of any un-associated sequences is indicative of a quantitative difference between the amount of the first and second target regions of nucleic acid in the sample.

The present invention relates to a method for detection of a quantitative difference between first and second target sequence regions present in a nucleic acid sample, use of the method, and a kit for carrying out the method of the invention.

The detection of small differences in nucleic acid content can be very important in detecting and diagnosing a disease or a predisposition to a disease. The differences may be differences in gene dosage or chromosome number. However the detection of small differences is very difficult.

Gene dosage differences in somatic cells are exemplified by chromosomal abnormalities resulting in gain or loss of genetic material often observed in malignant tumours. Among these abnormalities oncogene amplification (increase of gene copy number) is regarded as one of the most important mechanisms of oncogene activation in carcinogenesis. Successful detection of amplified genes can be useful diagnostically and appears to be especially important for predicting chemotherapy responsiveness in a number of malignancies. Correct selection of treatment strategies in individual cases can significantly improve life expectancy in patients with advanced tumours. The role of HER2/neu (c-erbB-2) gene amplification in determining breast cancer sensitivity to chemotherapy can serve as a good example.

However, in many cases it is difficult to reliably detect gene amplification in tumours since tumour tissue or cell samples obtained from patients are usually characterised by a strong presence of non-malignant cellular elements representing connective tissue, blood and lymphoid cells, inflammatory cells etc. For this reason analysis of gene amplification in this material requires high sensitivity, to allow detection of the presence of extra copies of target gene(s) in mixed samples of malignant and normal cells provided by surgical removal of tumours, biopsies, body fluid sampling etc.

Hereditary numerical chromosome abnormalities, that is an increase or decrease in the number of a particular chromosome, are known to cause a number of syndromes, which may lead to physical or mental disability in life (syndromes of Down, Klinefelter, Edward, Patau, XXX, XXY etc.). Therefore it is desirable to detect if an unborn child, in particular a foetus, has such an abnormality. Knowing the likelihood of disease can provide useful information to the parents who may wish to terminate the pregnancy or to prepare for caring for a disabled child.

Although cell free foetal DNA (cff DNA) is routinely used to determine foetal sex, for rhesus D status analysis, and for the diagnosis of rare genetic disorders e.g. β-thalassemia, no one has been able to apply this technology for the diagnosis of more common genetic conditions, due to the vast background of maternal cell free DNA. Some parties are attempting to either use SNPs in conjunction with foetal-specific cff RNA or foetal specific epigenetic markers e.g methylation, to select for foetal-specific cff DNA. However, SNPs can only be used to score foetuses that are heterozygous at the target SNP, so limiting the percentage of the population that can be tested. In addition, epigenetic markers are influenced by the exposure of the mother to environmental factors, which could in principle influence the epigenetic status of the foetus.

Conventional tests for chromosome abnormalities of foetuses are invasive because they require a medical professional to obtain cell material directly from the foetus or amniotic fluid surrounding the foetus. Such invasive procedures can lead to complications or even termination of the pregnancy.

It is desirable to provide a test for foetal numerical chromosome abnormality and/or gene dosage differences which is non-invasive, for example, by testing a sample of maternal blood, which will have foetal nucleic acid present in it. Foetal DNA can be found in maternal blood plasma early in pregnancy and its transplacental transition appears to be increased in cases of foetal chromosome abnormalities. However, it can be difficult to detect an imbalance in chromosome and/or gene copy numbers using maternal blood because the increased or decreased copy number of the target gene or chromosome observed in abnormal foetal DNA is likely to be masked by the presence of normal maternal DNA in excess. Conventional quantitative real time PCR is not sensitive enough to detect the small difference in gene or chromosome copy number, because only a small fraction of the template DNA is from the foetus.

The most common numerical chromosome abnormality known as Down's syndrome is caused by an imbalance in chromosome copy number where a patient has an additional copy of chromosome 21 (trisomy 21). A pure foetal DNA sample of a Down's syndrome afflicted subject would have 3:2 ratio of chromosome 21 to another chromosome, such as chromosome 10. Whereas in a maternal blood sample comprising a mixture of maternal and trisomic foetal DNA, the ratio of chromosome 21 to another chromosome would be much less at (2+x):2 (where x is a fraction of 1 corresponding to the share of trisomic foetal DNA in the sample), because the relative large amount of maternal DNA dominates the total DNA content of the maternal blood sample. For example, if foetal DNA provided 15% of the DNA in a maternal blood sample from a mother with a Down's syndrome-affected foetus, this would give the ratio of chromosome 21 to any other chromosome of 2.15:2. This ratio is far below the detection limit of conventional real time PCR. Thus, improved methods of DNA detection are required to observe these differences.

According to a first aspect of the invention, there is provided a method for the detection of a quantitative difference between the amount of a first target region of nucleic acid and a second target region of nucleic acid in a sample, comprising the steps of:
providing the sample comprising the nucleic acid;
amplifying the first and second target regions of the nucleic acid to obtain multiple copies of a first and a second sequence of nucleic acid;
associating the amplified first sequence with the amplified second sequence to form associated nucleic acid complexes which comprise the first sequence and the second sequence in a 1:1 ratio, wherein any excess of either the first sequence or the second sequence remain un-associated;
detecting any un-associated sequences, wherein detection of any un-associated sequences is indicative of a quantitative difference between the amount of the first and second target regions of nucleic acid in the sample.

Preferably the method of the invention comprises the step of eliminating the associated nucleic acid complexes prior to detecting the un-associated sequences.

According to another aspect of the present invention, there is provided a method for detection of an abnormality in a gene or chromosome copy number in a sample, comprising the steps of:
providing a sample comprising nucleic acid;
amplifying a first and a second target regions of the nucleic acid to obtain multiple copies of a first and a second sequence of nucleic acid;
associating the amplified first sequence with the amplified second sequence to form associated nucleic acid complexes which comprise the first sequence and the second sequence in a 1:1 ratio, wherein any excess of either the first sequence or the second sequence remain un-associated;
detecting any un-associated sequences, wherein detection of any un-associated sequences is indicative of a quantitative difference between the amount of the first and second target regions of nucleic acid in the sample, and wherein the detection of a quantitative difference is indicative of an abnormality in a gene or chromosome copy number.

Preferably the method of the invention comprises the step of eliminating the associated nucleic acid complexes prior to detecting the un-associated sequences.

Preferably the first target region is a gene or chromosome the copy number of which is to be studies, and the second target region is a different gene or chromosome, preferably the different gene or chromosome is present in normal copy number.

A method of the invention has an advantage that a non-invasive procedure can be used for a diagnosis of a gene or chromosome abnormality in a foetus using, for example, a maternal blood sample. Thus, carrying out the method of the invention does not increase the risk of termination of a pregnancy.

Detecting any un-associated sequences may comprise amplifying any un-associated sequences and detecting any amplification product, wherein detection of the amplification product, for example using real-time PCR, is indicative of a quantitative difference between the amount of the first and second target regions of nucleic acid in the sample.

Alternatively, un-associated sequences may be detected by incorporating a label into the sequences during the amplification and then detecting labelled products. The label may be a fluorophore included on one of the primers which remains a part of the un-associated sequences.

Alternatively or additionally unassociated DNA may be detected using one or more of the following methods: gel electrophoresis; incorporating a radioactive label; size fractionation; and mass spectrometry.

The term “associating” or “associated” is intended to describe the linking or binding of nucleic acid molecules. The association may be a hybridisation of the molecules of the first and second sequences. The association may be a direct covalent or non-covalent bond between the molecules, or an indirect binding of nucleic acid molecules whereby the nucleic acid molecules are each bound covalently or non-covalently to a linking molecule, such as another nucleic acid molecule.

It is understood that the term “quantitative difference” used herein refers to a difference in the number of copies of a gene, operon, chromosome, part of a chromosome, or a nucleic acid sequence, such as an amplification product. The term “quantitative detection” used herein refers to the detection of the difference in number of copies of a gene, operon, chromosome, part of a chromosome or a nucleic acid sequence, such as an amplification product.

The invention has an advantage in that a very small difference in gene or chromosome copy number can be detected. The invention may increase the sensitivity of quantitative detection of nucleic acid relative to standard PCR amplification methods by eliminating sequences which are not present in excess copy number, whilst isolating and amplifying sequences which are in excess relative to a normal sequence copy number.

The method advantageously may be applied to detecting quantitative differences in DNA sequence present in different groups of somatic cells within the same organism or in prenatal diagnosis of hereditary conditions.

The invention has an advantage in that the method is not dependent on SNPs or epigenetic modifications, and is hence not limited to a select percentage of the population.

The nucleic acid may be DNA or RNA, preferably DNA. The nucleic acid may be mixture of nucleic acid from malignant and normal/non-malignant tissue. The nucleic acid may be a mixture of nucleic acid from malignant and normal/non-malignant tissue present in a biopsy or body fluid sample. The nucleic acid may be a mixture of maternal and foetal nucleic acid. The nucleic acid may be a mixture of maternal and foetal nucleic acid found in a maternal blood sample.

The sample may be a tissue sample, such as a biopsy or tissue explant. The sample may comprise blood. The sample may, in one embodiment, comprise maternal blood. The sample may comprise body fluid. The sample may comprise blood plasma or serum. The sample may be maternal blood plasma, which comprises foetal nucleic acid. The sample may comprise maternal and foetal derived nucleic acid. The sample may be collected by a non-invasive procedure with respect to the foetus and/or the amniotic sac. The sample may be collected intravenously from a pregnant woman.

The sample may be obtained from amniotic fluid surrounding a foetus or embryo in utero, or directly from the foetus or embryo in utero. The sample may be obtained from a mammalian subject, preferably the sample is from a human.

An abnormality in a chromosome number may be an additional copy of a whole or part of a chromosome, or a missing copy of a whole or part of a chromosome. An additional copy number of a chromosome may be two or more copies, or three or more copies, of the chromosome or part of the chromosome, i.e. where the chromosome or part of the chromosome is in triplicate (also known in the art as a “trisomy”). For example, there are three copies of chromosome 21 in a subject with Down's Syndrome compared with two copies in a subject without Down's Syndrome. A missing copy of a chromosome may result in a single copy, or no copy, of a chromosome instead of the normal two copies in a healthy/un-afflicted subject (or one copy in the case of sex chromosomes in males).

An abnormality in a gene copy number in a subject may be one or more additional copies of a gene relative to the average copy number of the gene in a sample of subjects of a general population. An abnormality in a gene copy number in a subject may be one or more reductions in copy number of a gene relative to the average copy number of the gene in a sample of subjects of a general population. The sample of subjects of a general population may comprise one or more individuals who do not have symptoms of a disease associated with the gene.

Preferably, the first target region of the nucleic acid is associated with an abnormality, such as a disease, and the second target region of the nucleic acid is used as a control/standard.

The first target region may be a part of a gene, operon, or chromosome which is associated with a disease, disability or other clinical syndrome and the second target region may be part of a gene, operon, or chromosome which is used as a control/standard, which is known to be present in normal copy number, or vice versa.

The first target region of nucleic acid may comprise at least part of a region of a human chromosome selected from any of the group comprising chromosome number 1, 2, 3, 4, 5, 6, 7, 8, 9, 10, 11, 12, 13, 14, 15, 16, 17, 18, 19, 20, 21, 22, 23, the X chromosome, and the Y chromosome. Preferably the first target region of nucleic acid comprises a region of human chromosome selected from the group comprising human chromosome number 8, 9, 13, 16, 18, 21, and 22.

The first target region of nucleic acid may comprise a nucleic acid sequence of at least part of the HER2/neu (c-erbB-2) gene or at least part of the p53 gene. The first target region of nucleic acid may comprise a nucleic acid sequence of at least part of the c-myc gene, IL-6 gene, EGRF gene (Epidermal Growth Factor Receptor gene), BMI gene, or cadherin 7 gene. The first target region of nucleic acid may comprise a nucleic acid sequence of at least part of any of the group of genes comprising c-MYC, VEGFA, MMP9, PTEN, int-2/FGF3, KRAS, EBF1, IKZF1, GATA6, AKT2,MYB, SMAD4, CDKN2A, TOP2A, receptors for oestrogen, progesterone, HER1, uPAR, uPA, MET, RET, GLI, AKT2, CCND1 (cyclin D1), EGFR, ERBB2, MYCN, and MYCL1.

An advantage of the first target region of nucleic acid comprising a nucleic acid sequence of at least part of a specific gene, such as at least part of the HER2/neu (c-erbB-2) gene, is that mutations or changes in copy number of this gene may be detected. The HER2/neu (c-erbB-2) gene is implicated in some breast cancers. Meanwhile, the Myc family gene may be implicated in leukaemias, lung cancers, and breast cancers; the IL-6 gene and EGRF in neurological tumours; the BMI gene in lymphomas; and the cadherin 7 gene in prostate and testicular tumours.

The second target region of nucleic acid may comprise at least part of a region of a human chromosome of any of the group comprising chromosome number 1, 2, 3, 4, 5, 6, 7, 8, 9, 10, 11, 12, 13, 14, 15, 16, 17, 18, 19, 20, 21, 22, 23, the X chromosome, and the Y chromosome. The second target region of nucleic acid may comprise a region of human chromosome selected from the group comprising chromosome 2, chromosome 7, chromosome 9, chromosome 10, chromosome 11 and chromosome 14.

The second target region of nucleic acid may comprise a nucleic acid sequence of at least part of the housekeeping gene encoding glyceraldehyde-3-phosphate dehydrogenase (GAPDH) or Beta-actin.

The second target region of nucleic acid may comprise a nucleic acid sequence of at least part of a sequence that is normally in the same copy number as the first target region of nucleic acid. The second target region may be a highly conserved (for example, highly conserved in humans) sequence of nucleic acid.

The second target region of nucleic acid may comprise a nucleic acid sequence of any single copy sequence or gene that is not present on the same chromosome as the first target region, for example, SLIT1 or PI3KADP1.

The first and/or second target regions of nucleic acid may be between about 10 and about 8000 base pairs long; or between about 20 and about 2000 base pairs long; or between about 50 and about 1000 base pairs long. The first and/or second target regions of nucleic acid may be less than 500 base pairs long, alternatively less than 300 base pairs long, and alternatively less than 200 base pairs long. The first and/or second target regions of nucleic acid may be between about 100 and about 200 base pairs long. Preferably the first and second target regions of nucleic acid are similar in length, preferably within 10%, alternatively with 20%, of each other.

In an embodiment the first and second target sequences are different lengths, and this difference in length allows them to be distinguished from one another. Preferably the amplified first and second sequences are at least 5, 6, 7, 8, 9, 10 or more nucleotides different in size. Preferably the size difference is enough to allow them to be distinguished but not enough to affect the amplification rates.

The first and second target sequences when amplified may include different markers which allow them to be distinguished. For example, the first sequence may carry a first fluorophore and the second sequence may carry a second fluorophore, wherein the first and second fluorophore may be different. The fluorophore may be introduced on one or more the primers used to amplify the target sequence.

An advantage of selecting the length of the first and/or second target regions of nucleic acid is that the length of the regions can be selected or matched to provide a more equal amplification of these regions.

In some cases only part of a chromosome is in excess copy number (e.g. partial trisomy) or in reduced copy number (e.g. deletion or monosomy), thus, it is advantageous to select a particular region which is known to be in excess copy number or reduced copy number and causes symptoms of a chromosomal abnormality or related disease.

The detection of an abnormality in a chromosome copy number may comprise the detection of and/or diagnosis of a condition that is a hereditary numerical chromosome abnormality.

The detection of an abnormality in a chromosome copy number may comprise the detection of and/or diagnosis of a condition selected from the group comprising Down's Syndrome (Trisomy 21), Edward's Syndrome (Trisomy 18), Patau syndrome (Trisomy 13), Trisomy 9, Warkany syndrome (Trisomy 8), Cat Eye Syndrome (4 copies of chromosome 22), Trisomy 22, and Trisomy 16.

Additionally, or alternatively, the detection of an abnormality in a gene, chromosome, or part of a chromosome, copy number may comprise the detection of and/or diagnosis of a condition selected from the group comprising Wolf-Hirschhorn syndrome (4p-), Cri du chat syndrome (5p-), Williams-Beuren syndrome (7-), Jacobsen Syndrome (11-), Miller-Dieker syndrome (17-), Smith-Magenis Syndrome (17-), 22q11.2 deletion syndrome (also known as Velocardiofacial Syndrome, DiGeorge Syndrome, conotruncal anomaly face syndrome, Congenital Thymic Aplasia, and Strong Syndrome), Angelman syndrome (15-), and Prader-Willi syndrome (15-).

Additionally, or alternatively, the detection of an abnormality in the chromosome copy number may comprise the detection of and/or diagnosis of a condition selected from the group comprising Turner syndrome (Ullrich-Turner syndrome or monosomy X), Klinefelter's syndrome, 47,XXY or XXY syndrome, 48,XXYY syndrome, 49 XXXXY Syndrome, Triple X syndrome, XXXX syndrome (also called tetrasomy X, quadruple X, or 48, XXXX), XXXXX syndrome (also called pentasomy X or 49, XXXXX), and XYY syndrome.

Additionally, or alternatively, the detection of an abnormality in the gene or chromosome copy number may comprise the detection of and/or diagnosis of a condition selected from any of the group listed in Table 1.

TABLE 1
Chromosome Abnormalities and Disease

| Chromosome Abnormality | | Disease Association |
|---|---|---|
| X, | XO | Turner's Syndrome |
| Y | XXY | Klinefelter syndrome |
| | XYY | Double Y syndrome |
| | XXX | Trisomy X syndrome |
| | XXXX | Four X syndrome |
| | Xp21 deletion | Duchenne's/Becker syndrome, congenital |
| | | adrenal hypoplasia, chronic granulomatus |
| | | disease |
| | Xp22 deletion | steroid sulfatase deficiency |
| | Xq26 deletion | X-linked lymphproliferative disease |
| 1 | 1p- (somatic) | neuroblastoma |
| | monosomy | |
| | trisomy | |
| 2 | monosomy | |
| | trisomy 2q | growth retardation, developmental and |
| | | mental delay, and minor physical |
| | | abnormalities |
| 3 | monosomy | |
| | trisomy (somatic) | non-Hodgkin's lymphoma |
| 4 | monosomy | |
| | trsiomy (somatic) | Acute non lymphocytic leukaemia (ANLL) |
| 5 | 5p- | Cri du chat; Lejeune syndrome |
| | 5q- (somatic) | myelodysplastic syndrome |
| | monosomy | |
| | trisomy | |
| 6 | monosomy | |
| | trisomy (somatic) | clear-cell sarcoma |
| | 7q11.23 deletion | William's syndrome |
| | monosomy | monosomy 7 syndrome of childhood; |
| | | somatic: renal cortical adenomas; |
| | | myelodysplastic syndrome |
| | trisomy | |
| 8 | 8q24.1 deletion | Langer-Giedon syndrome |
| 8 | monosomy | |
| | trisomy | myelodysplastic syndrome; Warkany |
| | | syndrome; somatic: chronic myelogenous |
| | | leukemia |
| 9 | monosomy 9p | Alfi's syndrome |
| | monosomy | |
| | 9p partial trisomy | Rethore syndrome |
| | trisomy | complete trisomy 9 syndrome; mosaic |
| | | trisomy 9 syndrome |
| 10 | monosomy | |
| | trisomy (somatic) | ALL or ANLL |
| 11 | 11p- | Aniridia; Wilms tumor |
| | 11q- | Jacobson Syndrome |
| | monosomy (somatic) | myeloid lineages affected (ANLL, MDS) |
| | trisomy | |
| 12 | monosomy | |
| | trisomy (somatic) | CLL, Juvenile granulosa cell tumor (JGCT) |
| 13 | 13q- | 13q- syndrome; Orbeli syndrome |
| | 13q14 deletion | retinoblastoma |
| | monosomy | |
| | trisomy | Patau's syndrome |
| 14 | monosomy | |
| | trisomy (somatic) | myeloid disorders (MDS, ANLL, atypical |
| | | CML) |
| 15 | 15q11-q13 deletion | Prader-Willi, Angelman's syndrome |
| | monosomy | |
| | trisomy (somatic) | myeloid and lymphoid lineages affected, |
| | | e.g., MDS, ANLL, ALL, CLL) |
| 16 | 16q13.3 deletion | Rubenstein-Taybi |
| | monosomy | |
| | trisomy (somatic) | papillary renal cell carcinomas (malignant) |
| 17 | 17p- (somatic) | 17p syndrome in myeloid malignancies |
| | 17q11.2 deletion | Smith-Magenis |
| | 17q13.3 | Miller-Dieker |
| | monosomy | |
| | trisomy (somatic) | renal cortical adenomas |
| | 17p11.2-12 trisomy | Charcot-Marie Tooth Syndrome type 1; |
| | | HNPP |
| 18 | 18p- | 18p partial monosomy syndrome or Grouchy |
| | | Lamy Thieffry syndrome |
| | 18q- | Grouchy Lamy Salmon Landry Syndrome |
| | monosomy | |
| | trisomy | Edwards Syndrome |
| 19 | monosomy | |
| | trisomy | |
| 20 | 20p- | trisomy 20p syndrome |
| | 20p11.2-12 deletion | Alagille |
| | 20q- | somatic: MDS, ANLL, polycythemia vera, |
| | | chronic neutrophilic leukemia |
| | monosomy | |
| | trisomy (somatic) | papillary renal cell carcinomas (malignant) |
| 21 | monosomy | |
| | trisomy | Down's syndrome |
| 22 | 22q11.2 deletion | DiGeorge's syndrome, velocardiofacial |
| | | syndrome, conotruncal anomaly face |
| | | syndrome, autosomal dominant Opitz |
| | | G/BBB syndrome, Caylor cardiofacial |
| | | syndrome |
| | monosomy | |
| | trisomy | complete trisomy 22 syndrome |

The detection of an abnormality in a gene copy number may comprise the detection of and/or diagnosis of a cancer related condition.

The detection of an abnormality in a gene copy number may comprise the detection of and/or diagnosis of a condition selected from the group comprising breast cancer, leukaemia, lung cancer, neurological tumours, lymphomas, prostate cancer and testicular cancer.

Additionally, or alternatively, the detection of an abnormality in a gene copy number may comprise the detection of and/or diagnosis of a condition caused or associated with an additional copy number or reduced copy number of a gene.

The method of the invention may further comprise a step of whole genome amplification (WGA) prior to amplifying the first and second target regions of the nucleic acid.

An advantage of whole genome amplification is that a small nucleic acid sample may be amplified non-specifically, in order to generate a sample that is indistinguishable from the original but with a higher DNA concentration. This may allow fewer rounds of amplification of specific first and second target sequences thereby reducing the effects of differences in the efficiency of the amplification of different regions of DNA with different primers. Preferably, if WGA is used, 15 or less, preferably 10 or less, rounds of amplification of target regions is needed.

Whole genome amplification may be achieved using a commercially available kit, such as the GenomePlex® Whole Genome Amplification kit available from Sigma.

PCR (polymerase chain reaction) may be used for amplifying the first and second target regions of the nucleic acid to obtain multiple copies of a first and a second sequence of nucleic acid. Anti-sense/complementary nucleic acid sequences of the first and second sequences of nucleic acid may also be generated during the amplification step.

The first sequence may comprise the sequence of the first target region (or complement thereof), and an additional sequence provided by a primer. The second sequence may comprise the sequence of the second target region (or complement thereof), and an additional sequence provided by a primer.

The first and second target regions may be multiplied by amplification at a substantially equal rate, preferably at an equal rate. The skilled person will understand that minor differences in the amplification rate may be tolerated within error parameters which are readily determined by the skilled person.

In one embodiment, the first and second target regions may be multiplied by amplification at a substantially equal rate, with a difference in amplification rate of no more than 2%, e.g. a difference of 1.5% or less or 1% or less.

The concentration and/or choice of one or more primers, and/or other PCR constituents such as enzymes or dNTPs, may be adjusted to achieve a substantially equal rate of amplification.

The amplification at a substantially equal rate may be provided by amplifying sequences of substantially similar size, preferably with a difference in size of no more than 20%, such as a difference in size of 15% or less, 10% or less or 5% or less. The amplification at a substantially equal rate may be provided by providing primers with no homodimers or heterodimers. The amplification at a substantially equal rate may be provided by substantially matching the annealing temperatures of the primers for PCR amplification, e.g. having annealing temperatures within 5° C. of each other, such as within 2° C. of each other.

The first and second target regions may be simultaneously multiplied by amplification in the same reaction, or in a separate reaction. Amplification in the same reaction may be beneficial in ensuring that the conditions used to amplify the target (first target sequence) and reference (second target sequence) are identical, in order to minimise any differences between target and reference that may otherwise be caused by slight differences in the operational parameters.

The first and second target region amplification products may be mixed together immediately after amplification.

The first sequence of nucleic acid may be amplified from the nucleic acid sample using a first primer pair. The second sequence of nucleic acid may be amplified from the nucleic acid sample using a second primer pair.

The first and/or second sequence of nucleic acid, produced by amplification of the first and/or second target sequence, may comprise an additional sequence provided by a primer.

The first and/or second primer pair may comprise a forward primer. The first and/or second primer pair may comprise a reverse primer. The first and/or second primer pair may comprise a forward primer and a reverse primer.

The primers used all preferably include a sequence complementary to, or substantially complementary to, a flanking region of a target sequence to be amplified. In addition to the complementary region one or more of the primers may also include anchor regions. An anchor region is not complementary to a target sequence to be amplified but does become incorporated into the amplified product. The anchor may then in subsequent rounds of amplification be used as the starting point for amplification, with primers directed to the anchor being used rather than primers directed to a region of the target sequence. The advantage of this is that all sequences to be amplified can then have specific/predetermined primer recognition sequences which allow differences in amplification efficiency to be reduced. For example all sequences to be amplified may use the same anchor sequences or at least the same combination of anchor sequences. For example, the primer pair used to amplify the first target sequence may be the same as the primer pair used to amplify the second target sequence, the primers within each pair may be the same or different. If anchor sequences are used there may be a few rounds, say up to 10 rounds of amplification, and preferably less, with primers complementary to the target region, followed by more than 10, preferably more than 15, 20 or 25 rounds of amplification using primers directed to the incorporated anchor sequences.

In addition a sequence complementary to, or substantially complementary to, a flanking region of a target sequence to be amplified the primer may include the recognition sequence of a restriction enzyme. The restriction enzyme recognition sequence may be a non-palindromic sequence, such as that recognised by BstXI.

At least one primer of the first primer pair and/or second primer pair may comprise a sequence which forms part or all of a restriction enzyme recognition site, such that when the primers are used in amplification of the first and second target regions, the resulting first and second sequences comprise part or all of a restriction enzyme recognition site.

Preferably only one restriction enzyme recognition sequence is included. Preferably the same non-palindromic restriction enzyme recognition sequence is introduced into the first and second sequence such that upon cutting with the restriction enzyme the first and second sequences are left with complementary overhanging regions which allow the first and second sequences to associate by hybridisation.

An affinity tag may be provided on one or both primers of the primer pair. The affinity tag may be provided on the primer that is capable of hybridising with the sense strand of the first and/or second target region. The affinity tag may be provided on the primer that is capable of hybridising with the anti-sense/complementary strand of the first and/or second target region. Where both primers of the primer pair comprise an affinity tag, preferably the affinity tag on one primer is different to the other affinity tag on the other primer.

A benefit of providing an affinity tag on the primer that is capable of hybridising with the sense strand of the first and/or second target region is that after amplification, the anti-sense/complementary strands of the first and/or second nucleic acid sequences will be tagged, thus aiding their removal.

The affinity tags described herein may be of the biotin-avidin type, alternatively of the biotin-streptavidin type, a hybridisation sequence, for example comprising PNA, and/or DNA, or any other suitable affinity tag known to the skilled person.

A detectable label may be provided on one or both of the primers of a primer pair. The label may allow a sequence amplified with a particular primer or primer pair to be identified. If a different label is used on the primer pair used to amplify the first sequence than the label used on the primer pair used to amplify the second sequence, then the label may be used to determine the degree of amplification and/or to determine the amount of un-associated first and or second sequence. Alternatively, or additionally, the label may be used to allow an amplification product to be visualised. The label may be a fluorophore, for example, FAM—6-carboxyfluorescein or TET—tetrachlorofluorescein. Preferably the fluorophore is a labelled nucleotide located or near the 5′ end of the anchor.

Thus, in addition to the sequence complementary to, or substantially complementary to, a flanking region of a target sequence to be amplified, one or more of the primers may include one or more of the following: an anchor sequence; a sequence which on amplification forms a restriction enzyme recognition site; an affinity tag; and a detectable label.

In one embodiment the first primer pair may comprise a first tailed primer. The first tailed primer may comprise a complementary portion, which is substantially complementary to a sequence of at least part of the first target region, and a tail portion comprising a sequence which is substantially complementary to a sequence of at least part of the second target region, preferably complementary to the 3′ end of the sense strand of the second target region. The first primer pair may comprise a forward primer complementary to the 3′ end of the antisense strand of the first target region.

The second primer pair may comprise a second tailed primer. The second tailed primer may comprise a portion which is substantially complementary to a sequence of at least part of the second target region, and a tail portion comprising a sequence which is substantially complementary to a sequence of at least part of the first target region, preferably complementary to the 3′ end of the sense strand of the first target region. The first primer pair may comprise a forward primer complementary to the 3′ end of the antisense strand of the second target region.

The tail portion of the first and/or second tailed primer may be at the 5′ end of the primer.

The complementary portion of the first tailed primer may be complementary to a 3′ end of the first target region. The complementary portion of the second tailed primer may be complementary to a 3′ end of the second target region.

The complementary portion of the first tailed primer may be complementary to a 5′ end of the first target region. The complementary portion of the second tailed primer may be complementary to a 5′ end of the second target region.

The first sequence of nucleic acid may comprise a first association portion, which is complementary to at least part of the second sequence of nucleic acid. The second sequence of nucleic acid may comprise a second association portion, which is complementary to at least part of the first sequence of nucleic acid. At least part of the first association portion may be provided by the first tailed primer. At least part of the second association portion may be provided by the second tailed primer.

The association portion of the first and/or second sequence of nucleic acid may be at least 6 base pairs in length. The association portion of the first and/or second sequence of nucleic acid may be at least 10 base pairs, alternatively at least 20 base pairs in length, alternatively at least 40 base pairs in length. The association portion of the first and/or second sequence of nucleic acid may be between about 42 and about 60 base pairs in length.

The first association portion may be formed from the tail of the first tailed primer during amplification. The second association portion may be formed from the tail of the second tailed primer during amplification.

The sense strands of the first sequence and second sequence may be associated. The anti-sense/complementary strands of the first and second sequences may be removed prior to the association step.

Associating the first sequence with the second sequence to form the associated nucleic acid complex may be repeated at least once.

Associating the first sequence with the second sequence to form the associated nucleic acid complex may comprise ligating the first sequence to the second sequence.

A template nucleic acid may be provided to aid association of the first and second sequences. The template nucleic acid may be artificial, i.e. not found in nature, or synthesised for the purpose of the method herein. The template nucleic acid may comprise a first portion which is capable of hybridising to the first sequence. For example, the first portion of the template nucleic acid may be substantially complementary to all or part of the first sequence. The template nucleic acid may comprise a second portion which is capable of hybridising to the second sequence. For example, the second portion of the template nucleic acid may be substantially complementary to all or part of the second sequence.

The template nucleic acid may comprise DNA, or RNA, or PNA (peptide nucleic acid) or mixtures thereof. Preferably the template nucleic acid is DNA. The template nucleic acid may be affinity tagged, such as biotinylated.

The first and second sequences may be ligated directly to each other to form the associated nucleic acid complex.

Preferably a thermostable DNA ligase, such as Ampligase®, is used to ligate the first and second sequence, with or without a spacer sequence therebetween.

Using a thermo stable DNA ligase, such as Ampligase®, has an advantage that a higher hybridisation temperature can be used to ensure higher stringency, thus reducing the chances of non-specific binding to the template nucleic acid.

The template nucleic acid may provide a spacer portion between the first and second portions. Preferably the spacer portion acts as a template for polymerase activity/in-filling to form a spacer sequence between the first and second sequences when the first and second sequences are hybridised to the template nucleic acid. Alternatively, the spacer sequence may be provided as a pre-formed oligonucleotide which is complementary to the spacer portion.

The spacer portion and/or spacer sequence may provide a whole or part of a restriction recognition site, or a whole or part of an associated nucleic acid complex hybridisation sequence. The spacer portion and spacer sequence may together provide part of a double-stranded restriction enzyme recognition site.

Preferably the associated nucleic acid complex comprises a restriction enzyme recognition site. The associated nucleic acid complex may comprise two or more restriction recognition sites. The restriction enzyme recognition site(s) may be provided by at least part of the first sequence and/or at least part of the second sequence, or complementary sequences thereof. Preferably the restriction enzyme recognition site, or at least part of the restriction enzyme recognition site, is formed by a 3′ tail of the first sequence and by a 5′ tail of the second sequence.

Un-associated sequences and/or associated nucleic acid complex may be immobilised, for example on a bead. Immobilisation of the un-associated sequences and/or associated nucleic acid complex may be performed prior to an elimination step. Immobilisation may be via the affinity tag on the hybridised template nucleic acid.

In a further embodiment the amplified first and second sequences include a restriction enzyme recognition sequence or site which when cut allows the cut first and second sequences to associate. Preferably the restriction enzyme recognition sequence is introduced to the first and second sequences on the primers used to amplify the first and second target sequences. Preferably the restriction enzyme recognition sequence is non-palindromic and incorporated such that when cut the cut first sequence can associate only with the cut second sequence, and the cut second sequence can associate only with the cut first sequence. The restriction enzyme may be BstXI and the restriction enzyme recognition sequence may be:

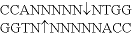

The term “substantially complementary” is intended to encompass sequences that are fully complementary (i.e. all bases pairs are complementary to the original sequence), or sequences that are partially complementary, but still capable of hybridisation with the original sequence (i.e. some base pairs may not be complementary but this does not prevent hybridisation).

The first and/or second sequence of nucleic acid may be less than about 500 base pairs in length, alternatively less than about 250 base pairs in length. The first and/or second sequence of nucleic acid may be less than about 100 base pairs in length.

The amplification may be symmetrical amplification and/or asymmetrical amplification. The amplification may comprise a symmetrical amplification step followed by an asymmetrical amplification step. The asymmetrical amplification may comprise the use of only one primer, preferably the forward primer. The sense strand may be favoured in the asymmetrical amplification step.

Advantageously asymmetric amplification allows selection of which strand to amplify. The sense strands of the target (first target sequence) and reference (second target sequence) may be favoured in the asymmetric amplification step because, for example, when they pair/associate, it is at their 3′ prime ends, such that their 3′OH groups can be extended by a polymerase. Asymmetric amplification will result in more sense strand, which is desirable.

The production of a single stranded amplification product may be favoured by purifying the sense strand from the antisense strand or vice versa, for example, by affinity tagging the antisense strand. The tailed primer may be affinity tagged. One strand may be removed by digestion, for example, with lambda exonuclease.

Beads, preferably magnetic beads, may be used to remove or purify nucleic acid using an affinity tag anchored thereon. The affinity tag may be of the biotin-avidin type, alternatively of the biotin-streptavidin type, or a hybridisation sequence, for example comprising PNA, and/or DNA, or any other suitable affinity tag known to the skilled person.

The step of associating the first sequence with the second sequence to form the associated nucleic acid complex may be repeated at least once. Repetition of the association step may advantageously reduce the error rate for incorrect hybridisation/association.

Associating the first sequence with the second sequence to form the associated nucleic acid complex may comprise hybridising the first sequence of nucleic acid to the second sequence of nucleic acid.

In an embodiment in which tailed primers have been used to introduce an association portion to the first and second sequences, associating the first sequence with the second sequence to form the associated nucleic acid complex may comprise hybridising the first association portion on the first sequence of nucleic acid to the second association portion on the second sequence of nucleic acid.

The associated nucleic acid complex may be at least partially double stranded. The hybridised first and second sequences of nucleic, which form the associated nucleic acid, may be extended by treatment with a polymerase or by the use of residual polymerase activity in the preceding amplification (such as PCR), in order to form a (fully) double stranded associated nucleic acid complex.

The associated nucleic acid complex may be cross-linked, for example using a chemical cross-linker such as Mitomycin C. The chemical cross-linker may be used in combination with catalysts, e.g. one or more enzymes and co-enzymes as catalysts, for example, DT Diaphorase and NADH. The cross-linking may suitably be carried out under aerobic conditions.

An advantage of providing fully-duplexed (fully double stranded) associated nucleic acid complex is that it is more stable than partially-duplexed (partially double stranded) associated nucleic acid complex. Thus, it is likely to be more stable when cross-linked, and act as a better product for digestion by restriction enzymes, or nucleases, such as double stranded DNA specific nucleases (DSN).

The associated nucleic acid complex may comprise a nuclease and/or restriction enzyme recognition site. The associated nucleic acid complex may comprise at least two restriction recognition sites. The restriction enzyme recognition site(s) may be provided by at least part of the first sequence and/or at least part of the second sequence, or complementary sequences thereof.

In an embodiment where the associated nucleic acid complex is eliminated this may comprise cutting the associated nucleic acid complex at one or more positions to form truncated fragments. Preferably the associated nucleic acid is cut at one or more positions. The cutting may be carried out using a restriction enzyme which recognises the restriction recognition site(s). Alternatively, or additionally, the cutting may comprise treating the associated nucleic acid with a nuclease.

Where “elimination” is described herein, it is intended to describe the truncation or fragmentation of the associated nucleic acid complex, such that the first and/or second sequences contained therein are truncated or fragmented; or the substantial or complete removal of the associated nucleic acid complex; or complete or partial degradation of the associated nucleic acid complex; or substantial or complete purification of un-associated sequence from the associated nucleic acid complex. The elimination step may be repeated as many times as necessary to obtain the desired purity of un-associated sequence and/or the desired amount of elimination of the associated nucleic acid complex, which can be readily determined by the skilled person.

The cutting of the associated nucleic acid complex may be at one or more positions within the first and/or second sequence of nucleic acid which is hybridized to the template nucleic acid, such that the first and/or second sequence of nucleic acid is truncated. Preferably the cutting reduces the size of the second sequence by at least 5, 10, or 12 base pairs. The cutting may disrupt primer recognition sequences on the first and/or second sequence.

The restriction enzyme may cut the associated nucleic acid complex at least once at flanking regions of the restriction recognition sequence. The restriction enzyme may cut the associated nucleic acid complex at least 5 or 10 base pairs away from the restriction recognition sequence, in particular towards the 3′ end of the ligated sequence.

The restriction recognition sequence may comprise 5′-ACNNNNNCTCC-3′ representing the recognition sequence of BsaX I. BsaX I may excise the following fragment:

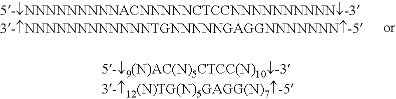

The restriction enzyme may be selected from any of the group comprising:

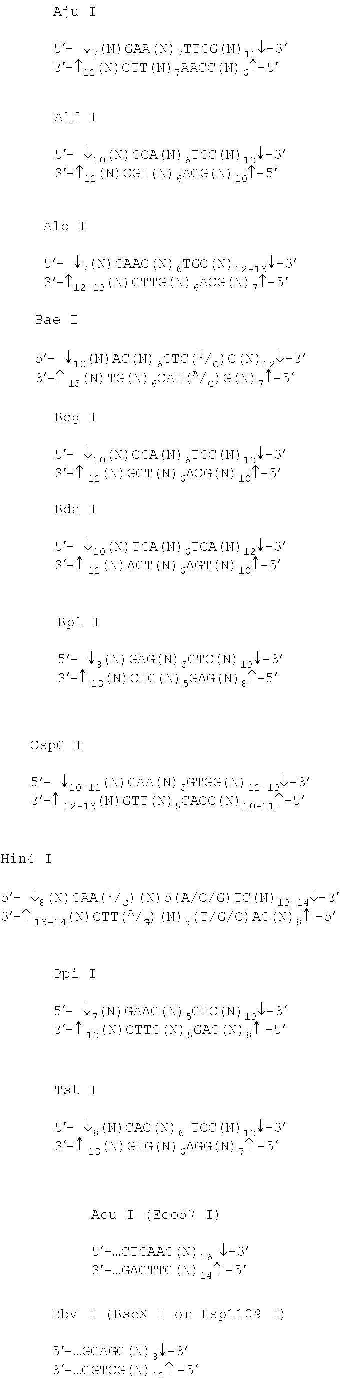

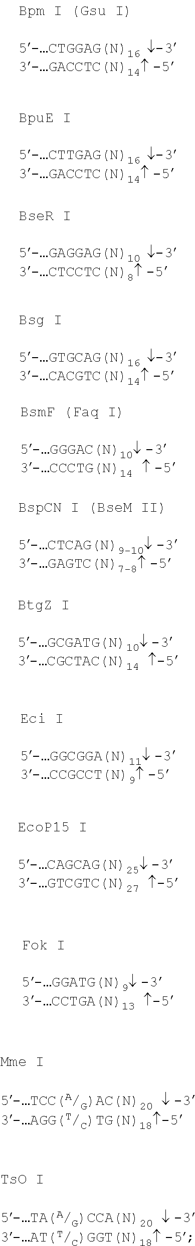

and

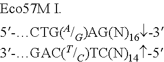

Preferably, following elimination of the associated nucleic acid complex, the un-associated nucleic acid is amplified. Amplification of the un-associated nucleic acid may be by polymerase chain reaction (PCR) or real-time PCR (rt-PCR). Preferably the amplification of un-associated nucleic acid or un-associated first sequence is by real-time PCR. In an embodiment where real-time PCR is used, the real-time PCR primers may have a fluorescence marker to enable the detection of polymerisation. Preferably fluorescent reporter molecule-linked primers, such as Scorpion® primers, are used in the real-time PCR amplification.

TaqMan® may be used for real time PCR. A Taqman® assay, or similar multi-well array, may be used in the step of amplification of un-associated nucleic acid. A TaqMan® array, or similar multi-well array, may be used to perform multiple (i.e. at least two, or three) and simultaneous reactions according to the method of the invention. The same sample may be used in the array, where the first and/or second target regions are different in each reaction (e.g. each reaction investigates a different gene or chromosome copy number). Alternatively, different samples (e.g. from different subjects) may be used in the array, where the first and/or second target regions are the same for each reaction.

Preferably the amplification of the un-associated nucleic acid sequence is exponential.

Preferably detecting any amplification product is quantitative detection.

Detecting any amplification product may comprise gel electrophoresis of the amplification product, or detecting fluorescence markers or probes during and/or after the amplification. Preferably the detection of the amplification product is quantitative detection during the amplification.

Preferably the detection of amplified first sequence in the amplification product is indicative of an excess copy number of the first target region. Detection of amplified second sequence in the amplification product may be indicative of a reduced copy number of the first target region.

Following elimination of the associated nucleic acid complex, any truncated first and/or second sequence of nucleic acid may be amplified. Preferably the amplification of the truncated first and/or second sequence of nucleic acid is linear.

The truncated first and/or second sequence may be amplified linearly because only one of the primers has a complementary primer binding site, the other primer binding site being eliminated in the elimination step. Whereas, any un-associated nucleic acid sequence may be amplified exponentially, because both primer binding sites remain intact. This has an advantage that the amplification product from the un-associated nucleic acid sequence is detectable in relatively large quantities and amplification of other truncated products is negligible, thus, providing a clearer result.

A third set of primers may be used for amplification of the un-associated first sequence of nucleic acid. The third set of primers may be used for amplification of any truncated first nucleic acid sequence. In an embodiment where a third set of primers is used for amplification of any truncated first nucleic acid sequence, preferably only one primer of the third set of primers will be capable of binding to the truncated first sequence. Preferably, the third set of primers are capable of hybridising to the same sequence of nucleic acid as the first primer pair.

A fourth set of primers may be used for amplification of any truncated second sequence. In an embodiment where a fourth set of primers is used for amplification of any truncated second nucleic acid sequence, preferably only one primer of the fourth set of primers will be capable of binding to the truncated second sequence. Preferably, the fourth set of primers are capable of hybridising to the same sequence of nucleic acid as the second primer pair.

In another embodiment, the un-associated nucleic acid may be detected without amplification by using a probe, such as a fluorescence probe. Alternatively un-associated nucleic acid may be detected on the basis of size, for example, by using a fragment analyser or by using gel electrophoresis. Preferably un-associated first and second nucleic acid sequences are different sizes. Alternatively the first and second nucleic acids may incorporate a different detectable label, this would allow the different nucleic acids to be distinguished if they were the same or different sizes.

The method of the invention may further comprise the detection of a quantitative difference between the amount of one or more additional first target regions of nucleic acid, that is one or more target regions in a region which may have a potential increase or decrease in copy number relative to the amount of the second target region, and optionally one or more additional second (reference) target regions.

In addition to the second target region, one or more additional reference target regions may be used in the method of the invention.

The one or more additional target regions of nucleic acid, in addition to the first target region, may comprise any of the optional features described herein with reference to the first target region of nucleic acid. The one or more additional target regions of nucleic acid may be amplified to form one or more additional sequences of nucleic acid. The one or more additional sequences of nucleic acid may comprise any of the optional features described herein with reference to the first sequence of nucleic acid.

The one or more additional target regions of nucleic acid in addition to the second (reference) target region, may comprise any of the optional features described herein with reference to the second target region of nucleic acid. The one or more additional target regions of nucleic acid may be amplified to form one or more additional sequences of nucleic acid. The one or more additional sequences of nucleic acid may comprise any of the optional features described herein with reference to the second sequence of nucleic acid.

Preferably, in a method of the invention, an equal number of first target regions and second target regions are used. Alternatively there are more first target regions than second target regions, or there may be more second target regions than first target regions. For example, there may be two or more first and/or second target regions, there may be three or more first and/or second target regions, there may be four or more first and/or second target regions, and there may be five or more first and/or second target regions.

The terms first target sequence and first target region are used interchangeably herein and intended to have the same meaning. Similarly, the terms second target sequence and second target region are used interchangeably herein and intended to have the same meaning.

According to another aspect of the invention, there is provided a method for detection of a quantitative difference between a first target region of nucleic acid and a second target region of nucleic acid in a sample, comprising the steps of:
providing the sample comprising nucleic acid;
amplifying by PCR a first target region and a second target region of the nucleic acid, such that multiple copies of a first species and a second species of double-stranded nucleic acid are formed, wherein the amplification uses primers and one or more of the primers comprise part of a restriction enzyme recognition site;
purifying either the sense or antisense strands of the first and second species of double-stranded nucleic acid to form a first sequence and a second sequence of single-stranded nucleic acid, wherein the first sequence and/or the second sequence comprise part of a restriction enzyme recognition site;
annealing the first sequence and second sequence to a template nucleic acid, the template nucleic acid comprising:
   a first complementary region which has a sequence substantially complementary to the first sequence,
   a second complementary region which has a sequence substantially complementary to the second sequence;
ligating the annealed first sequence to adjacent annealed second sequence to form a double-stranded nucleic acid complex comprising a restriction enzyme recognition site, wherein any excess of the first sequence relative to the second sequence results in a partially double-stranded nucleic acid complex comprising a double-stranded portion where the first sequence is annealed to the template nucleic acid, and a single-stranded portion which is complementary to the second sequence;
immobilising the double-stranded and partially double stranded nucleic acid complexes as well as all remaining artificial nucleic acid template on a solid surface such as magnetic beads;
cutting the double-stranded nucleic acid complex to form restriction fragments using a restriction enzyme which recognises the restriction enzyme recognition site, wherein a proportion of the restriction fragments comprise a truncated first sequence and/or a proportion of restriction fragments comprise a truncated second sequence;
recovering any annealed first sequence from the partially double-stranded nucleic acid complex by denaturing the partially double-stranded nucleic acid complex and removing the template nucleic acid;
amplifying any recovered first sequence to form an amplification product; and
detecting any amplification product; wherein detection of the amplification product of the first sequence is indicative of quantitative difference between the first target region of nucleic acid and the second target region of nucleic acid in the sample.

The template nucleic acid may be removed by immobilisation, for example on a bead.

The step of amplifying any recovered first sequence to form an amplification product may further comprise a control step of amplifying, or attempting to amplify any other nucleic acid sequence that may be present, such as the second sequence, alternatively any truncated first and truncated second sequence that may be present.

Preferably the detection of amplified first sequence in the amplification product is indicative of an excess copy number of the first target region.

The first target region may be a part of a gene, operon, or chromosome which is associated with a disease, disability or other clinical syndrome and the second target region may be part of a gene, operon, or chromosome which is used as a control/standard, which is known to be present in normal copy number, or vice versa.

Preferably detecting any amplification product is quantitative detection.

The recovered excess first sequence may be amplified exponentially, such that an abundance of amplification product is formed and can be detected.

Truncated first sequence and/or second sequence may also be recovered by denaturing the cut double-stranded nucleic acid complex. The truncated first sequence and/or second sequence may be amplified. Preferably the truncated first sequence and/or second sequence is linearly amplified such that only low levels of amplification product are detected.

The restriction enzyme recognition site(s) may be provided by at least part of the first sequence and/or at least part of the second sequence. Preferably the restriction enzyme recognition site, or at least part of the restriction enzyme recognition site, is formed by a 3′ tail of the first sequence and a 5′ tail of the second sequence.

Purification and/or removal of nucleic acid may be aided by the use of an affinity tag on the nucleic acid to be removed and/or purified. Beads, preferably magnetic beads, may be used to remove or purify nucleic acid using an affinity tag anchored thereon.

According to a yet further aspect of the invention, there is provided a method for detection of a quantitative difference between a first target region of nucleic acid and a second target region of nucleic acid in a sample, comprising the steps of:
providing the sample comprising nucleic acid;
amplifying by PCRₐ first target region using a first primer pair to form a double stranded first nucleic acid sequence and a second target region using a second primer pair to form a double stranded second nucleic acid sequence wherein the first primer pair comprises:
   a first tailed primer comprising a complementary portion, which is substantially complementary to a sequence of at least part of the first target region, and a tail portion comprising a first association sequence which is substantially complementary to a sequence of at least a part of the second target region, and a second primer complementary to the other strand of the first target region; and wherein the second primer pair comprises:
   a second tailed primer comprising a complementary portion, which is substantially complementary to a sequence of at least part of the second target region, and a tail portion comprising a second association sequence which is substantially complementary to a sequence of at least a part of the first target region, and a second primer complementary to the other strand of the second target region;
amplifying only one strand of the double-stranded first and second nucleic acid sequences by asymmetric PCR
hybridising the amplified single stranded first nucleic acid sequence and the amplified single stranded second nucleic acid sequence using the first and second association sequence to form an associated nucleic acid complex in which the first and second sequences are associated in a 1:1 ratio;
using a polymerase to form a substantially fully double stranded double stranded nucleic acid complex;
eliminating double stranded DNA;
amplifying any remaining single stranded DNA;
detecting the presence of any amplified DNA.

Wherein detection of an excess of the first nucleic acid sequence may be indicative of an increase in copy number of the gene, operon or chromosome from which the first target region is taken. Wherein detection of an excess of the second nucleic acid sequence may be indicative of a decrease in copy number of the gene, operon or chromosome from which the first target region is taken.

The first target region may be a part of a gene, operon, or chromosome which is associated with a disease, disability or other clinical syndrome and the second target region may be part of a gene, operon, or chromosome which is used as a control/standard, which is known to be present in normal copy number, or vice versa.

In an alternative embodiment of this aspect of the invention the amplification of remaining single stranded DNA may be omitted if the single stranded DNA present can be detected without amplification, for example, the inclusion of a detectable marker in the single stranded DNA may be sufficient to allow a quantitative difference between the amounts of single stranded DNA of the first nucleic acid sequence and single stranded DNA of the second nucleic acid sequence to be determined.

It will be appreciated that optional features applicable to other aspects of the invention may be used with this aspect of the invention.

According to a further aspect of the invention, there is provided a method for detection of a quantitative difference between a first target region of nucleic acid and a second target region of nucleic acid in a sample, comprising the steps of:
providing the sample comprising nucleic acid;
amplifying by PCR a first target region and a second target region of the nucleic acid, such that multiple copies of a first species comprising a first nucleic acid sequence and a second species comprising a second nucleic acid sequence of double-stranded nucleic acid are formed, wherein the amplification uses primers which introduce a restriction enzyme recognition site into the double stranded nucleic acid sequences produced;
cutting the double-stranded nucleic acid species using a restriction enzyme which recognises the introduced restriction enzyme recognition site;
annealing the cut first nucleic acid sequence to the cut second nucleic acid sequence in a 1:1 ratio, wherein any excess of the first or second nucleic acid sequence remains un-annealed;
detecting the amount of the un-annealed nucleic acid sequences present, wherein a difference in the level of the first nucleic acid sequence relative to the second nucleic acid sequence indicates a quantitative difference between the amount of the first target region in the sample and the second target region in the sample.

Preferably the first and second sequence are annealed by first hybridising complementary overhanging ends and then ligating the hybridised sequences.

Preferably when detecting the amount of the un-annealed nucleic acid sequences, un-annealed first nucleic acid sequences can be distinguished from un-annealed second nucleic acid sequences. The amount of annealed nucleic acid sequences may also be detected.

An excess of the first nucleic acid sequence may be indicative of an increase in copy number of the gene, operon or chromosome from which the first target region is taken.

An excess of the second nucleic acid sequence may be indicative of an decrease in copy number of the gene, operon or chromosome from which the first target region is taken.

The amount of annealed and/or un-annealed nucleic acid may be determined by gel electrophoresis and/or by a fragment analyser and/or by any other suitable method.

The first target region may be a part of a gene, operon, or chromosome which is associated with a disease, disability or other clinical syndrome and the second target region may be part of a gene, operon, or chromosome which is used as a control/standard, which is known to be present in normal copy number, or vice versa.

The restriction enzyme recognition site may be arranged such that when cut the first nucleic acid sequence can anneal only to the cut second nucleic acid and not to other cut first nucleic acid sequences. Similarly, the restriction enzyme recognition site may be arranged such that when cut the second nucleic acid sequence can anneal only to the cut first nucleic acid and not to other cut second nucleic acid sequences. The restriction enzyme recognition site may be palindromic.

The primers used may also comprise a detectable label. The detectable label may be a fluorophore. Preferably is a fluorophore is used it is retained on the nucleic acid after it has been cut with a restriction enzyme. Preferably the detectable label is introduced on a primer that does not introduce the restriction enzyme recognition sequence.

The primers used may also include an anchor sequence. The anchor sequence may be the same on all primers used. Alternatively, the anchor sequence may be different on all the primers used. Alternatively, the anchor sequence may be the same in each primer pair used, however each primer in each pair may have a different anchor sequence.

The nucleic acid in the sample may be first amplified by whole genome amplification prior to amplification of the specific first and second target regions.

The method of the invention may include amplifying and detecting the presence of further target sequences/regions in addition to the first and second target regions. In one embodiment more than one first target region is amplified, that is more than one region which may be involved in the disease or condition of interest may be amplified, for example, in the case of a test for Down's syndrome more than one target sequence on chromosome 21 may be considered. Alternatively, or in addition, more than one second or reference regions may be amplified and detected. Preferably where more than one first or second target region is amplified at least two first and/or second target sequences are amplified, preferably at least three, four, five, six, seven, eight or more first and/or second target sequences are amplified

It will be appreciated that optional features applicable to other aspects of the invention may be used with this aspect of the invention.

The invention provides the use of the method of the invention, as described above, to determine if an individual has an increase or decrease in gene or chromosome copy number.

According to another aspect of the invention, there is provided a method of diagnosis to determine if an individual has an increase or decrease in gene or chromosome copy number by carrying out any method of the invention herein.

According to another aspect of the present invention, there is provided the use of any method of the invention to detect an abnormality in a gene or chromosome copy number in a sample for at least two different genes or chromosomes.

The method may be used to detect an abnormality in a gene or chromosome copy number for at least three, four, five, six or more different genes or chromosomes. An array, for example a multiwall array may be used. The array may comprise a Taqman® array, or similar simultaneous PCR multi-well array system.

According to another aspect of the invention, there is provided a use of any method according to the method herein, to determine if an individual has an increase or decrease in gene or chromosome copy number.

The term “diagnosis” used herein refers to the ability to demonstrate an increased likelihood that a subject has, or does not have, a specific condition or conditions or that an existing condition or conditions have certain specific characteristics such as therapy sensitivity/resistance.

According to another aspect of the invention, there is provided a use of the method of any other aspect of the invention to determine the choice of treatment for a condition.

The condition may be cancer. The choice of treatment may be a choice of chemotherapy regime and/or agent.

According to another aspect of the invention, there is provided a kit comprising one or more primers suitable for carrying out the method according to the invention herein and instructions.

The kit may further comprise a nuclease and/or a restriction enzyme suitable for carrying out the method of the invention herein.

The kit may further comprise beads, preferably magnetic beads. The beads may comprise an affinity-tag or oligonucleotide anchored thereon.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

An embodiment of the present invention will now be described herein, by way of example only, with reference to the following figures.

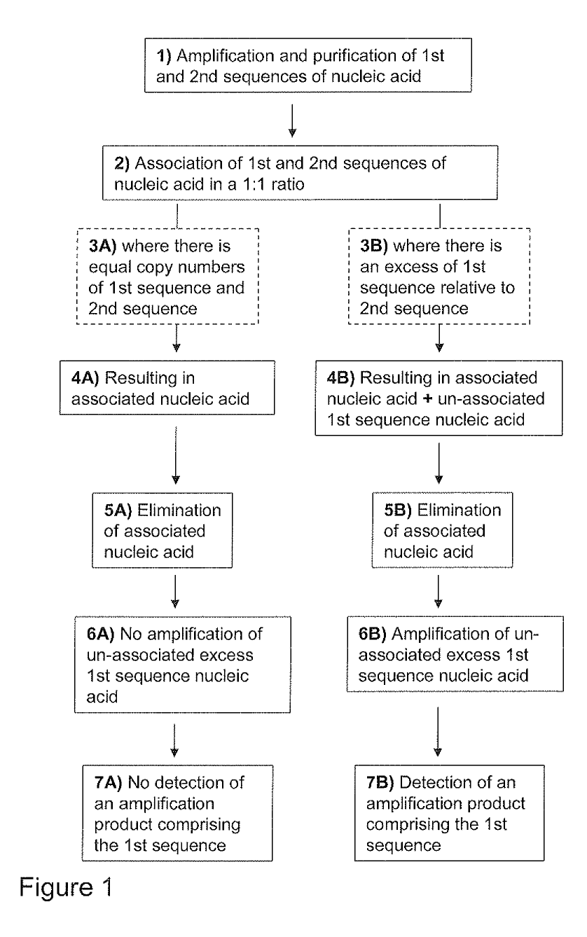

FIG. 1—illustrates general steps of a first embodiment of the invention;

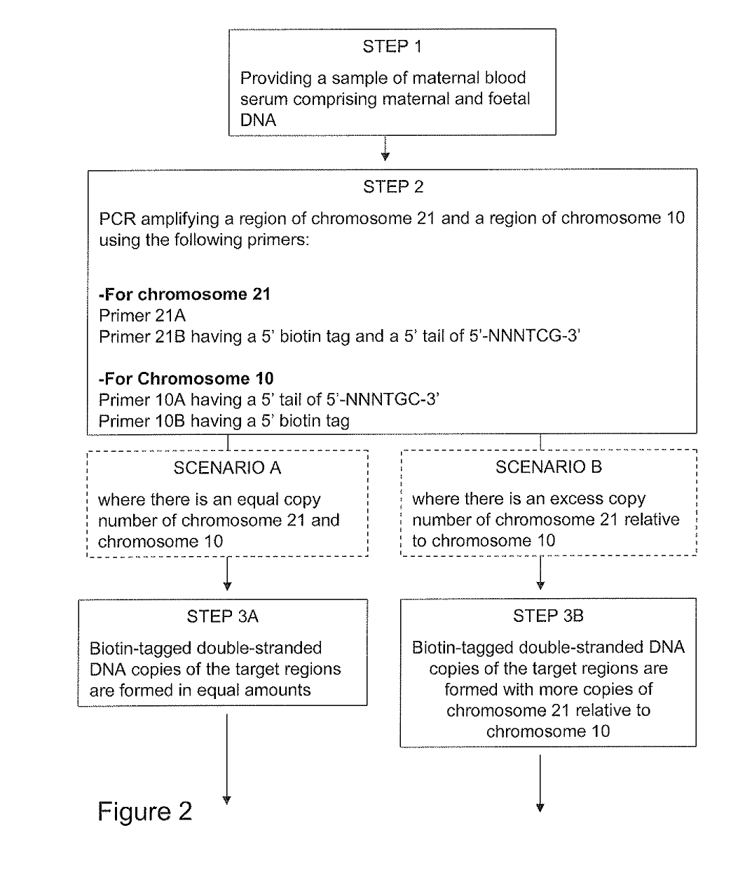

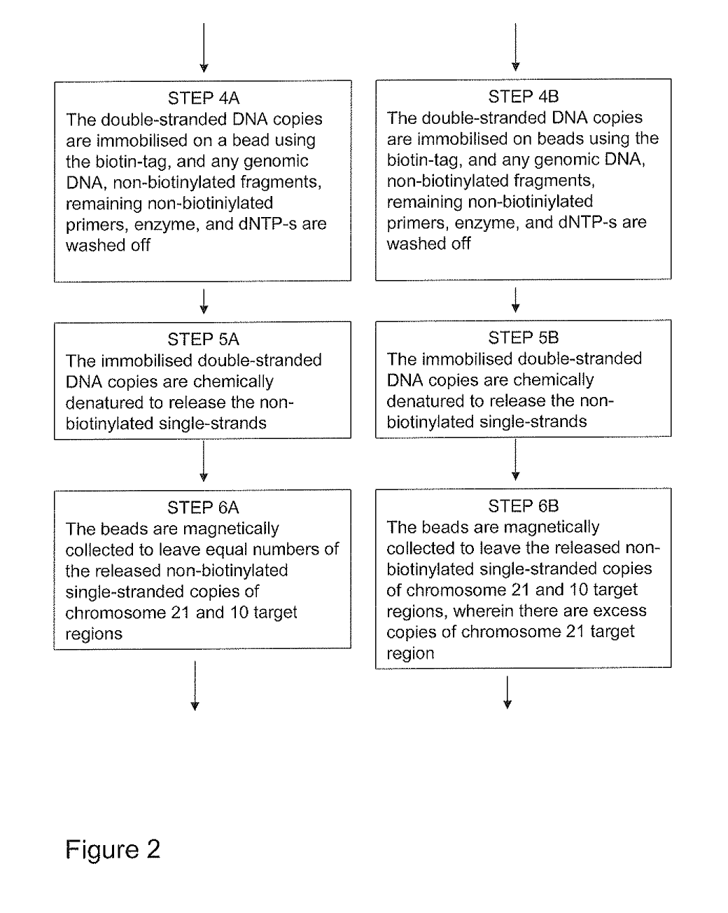

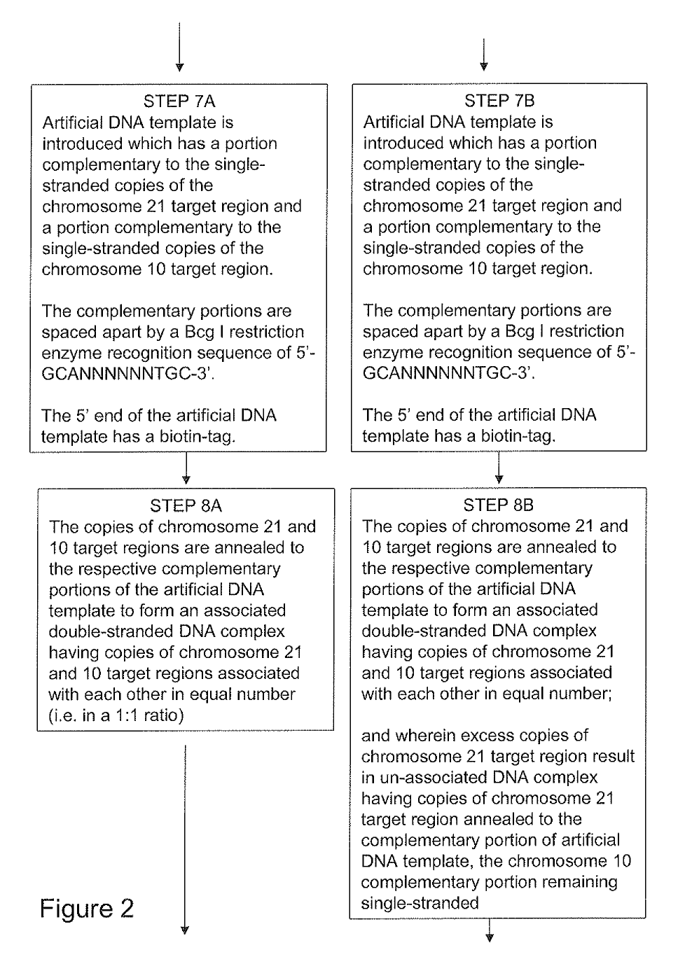

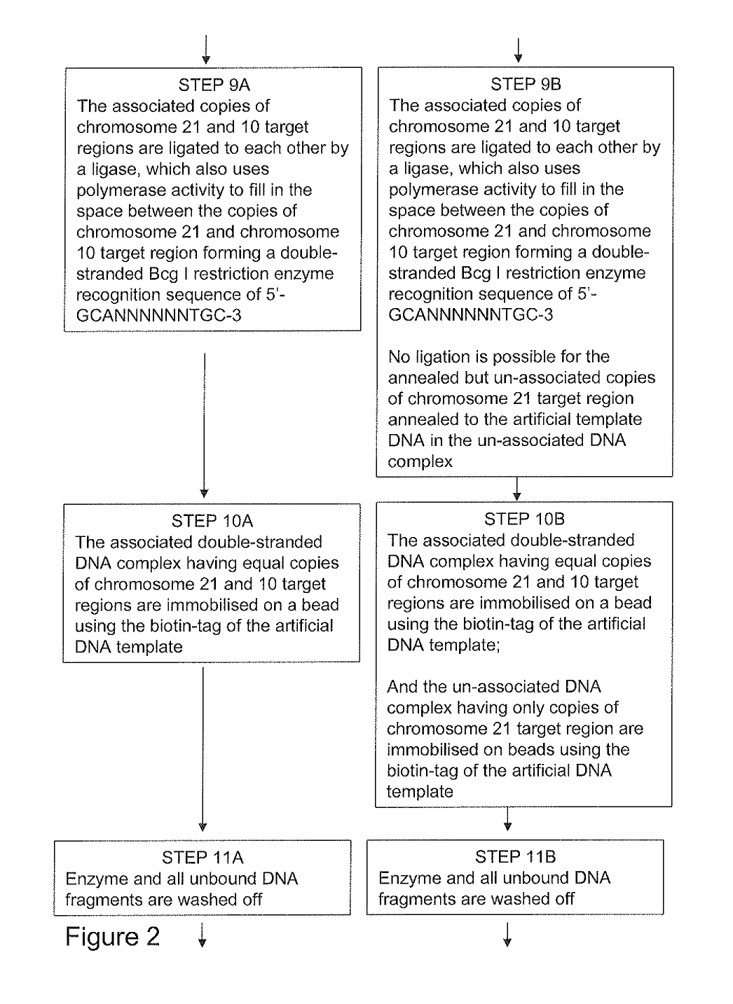

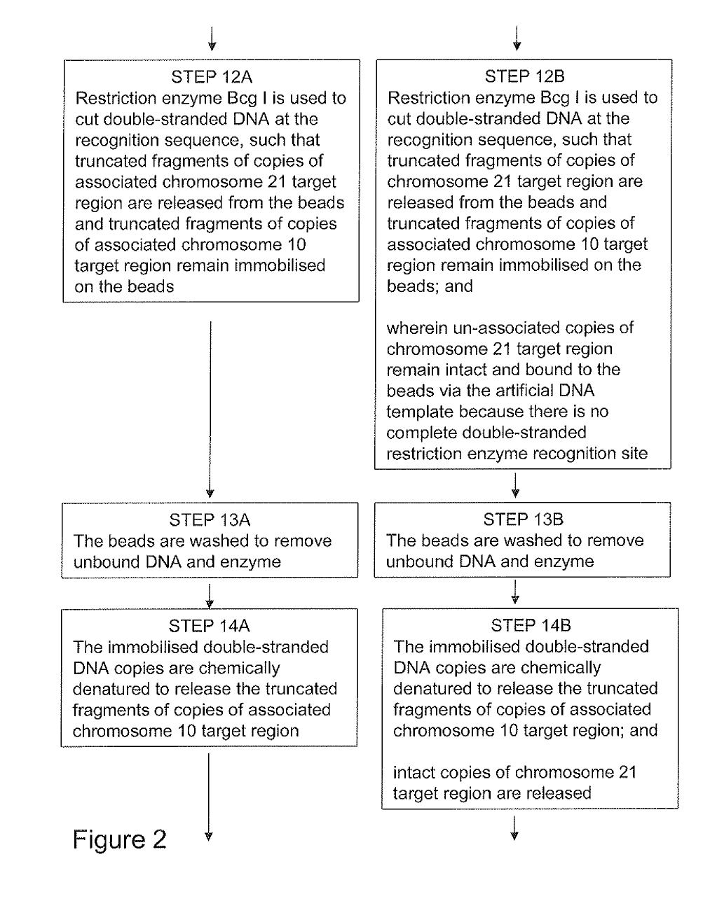

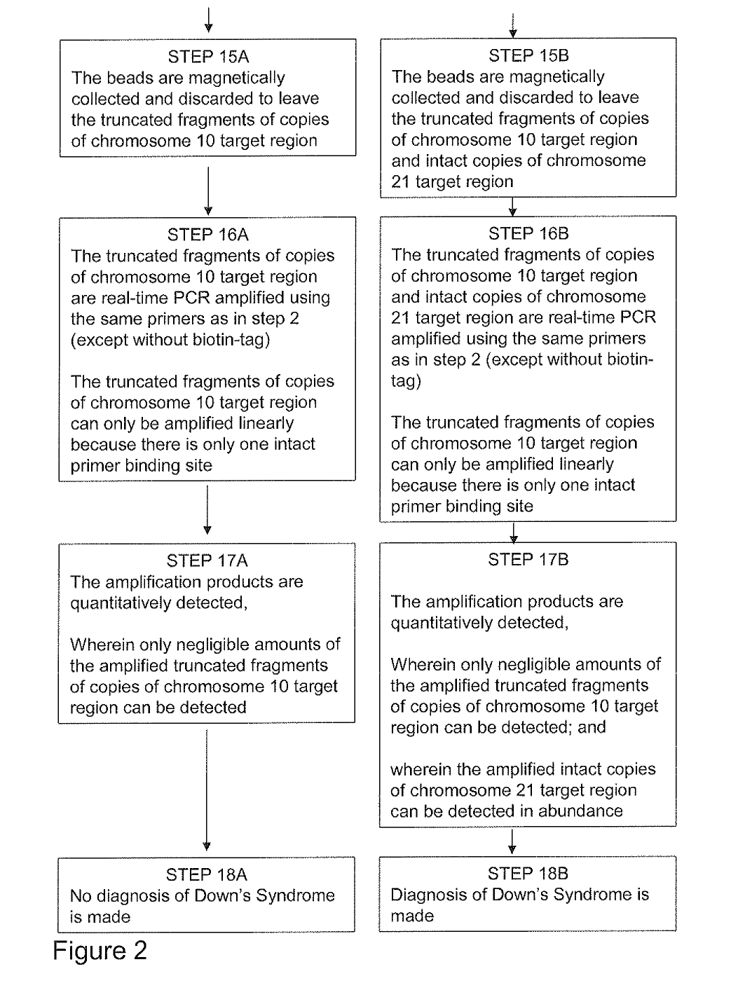

FIG. 2—illustrates steps of the first embodiment of the invention in more detail than illustrated in FIG. 1;

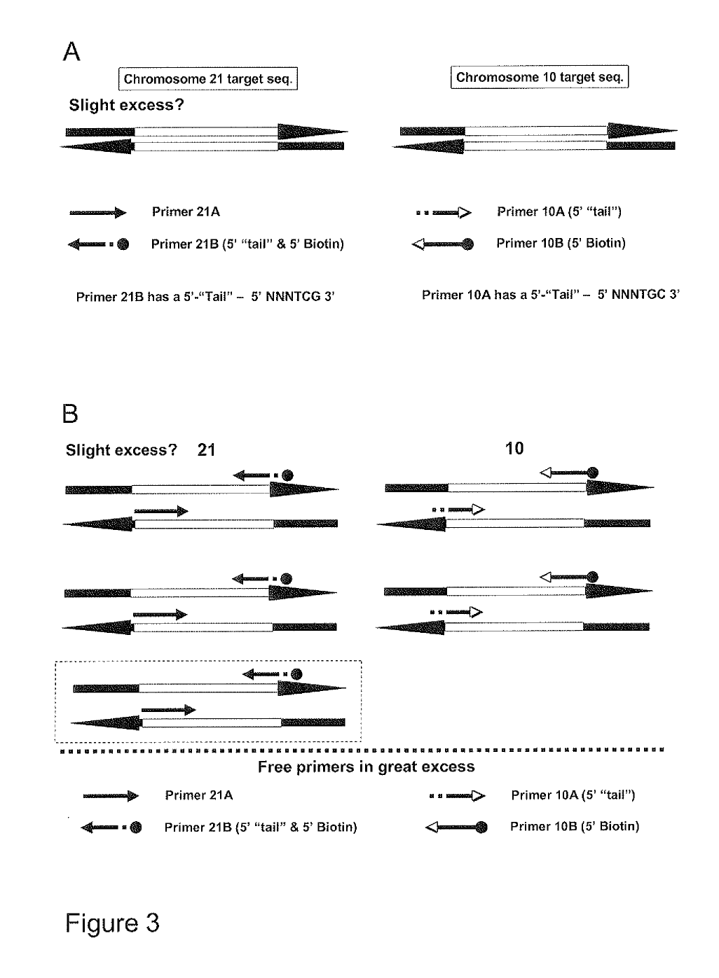

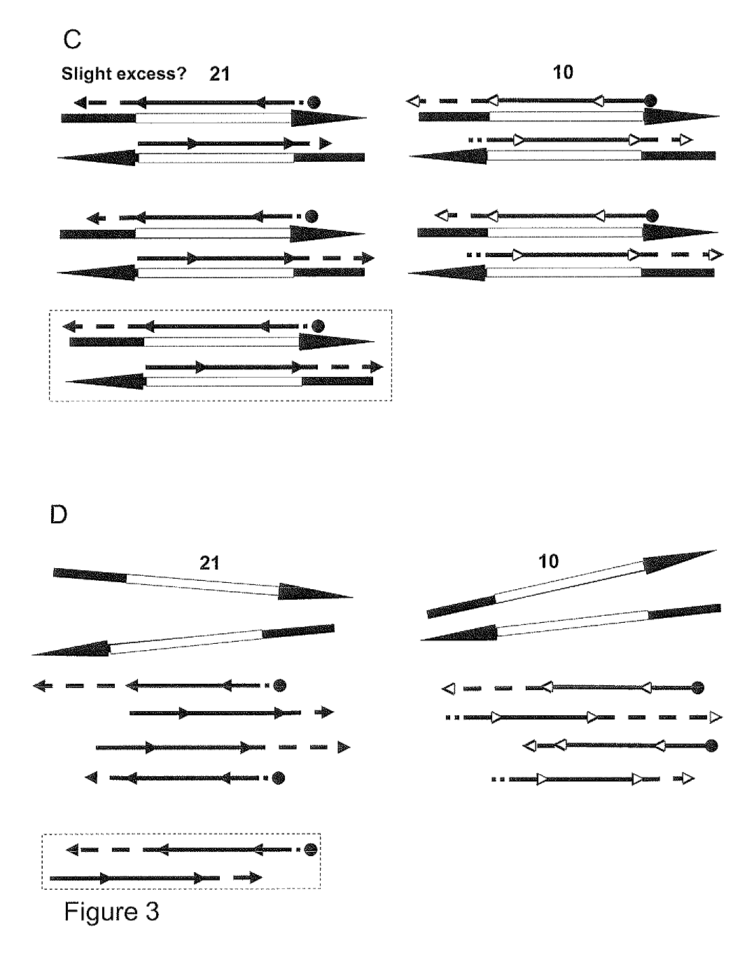

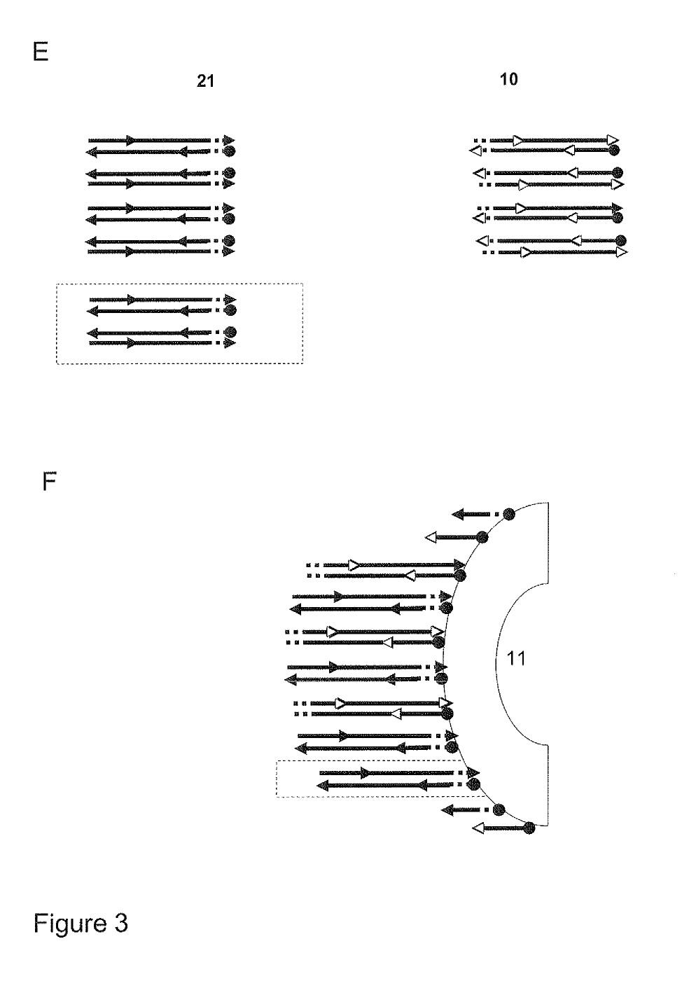

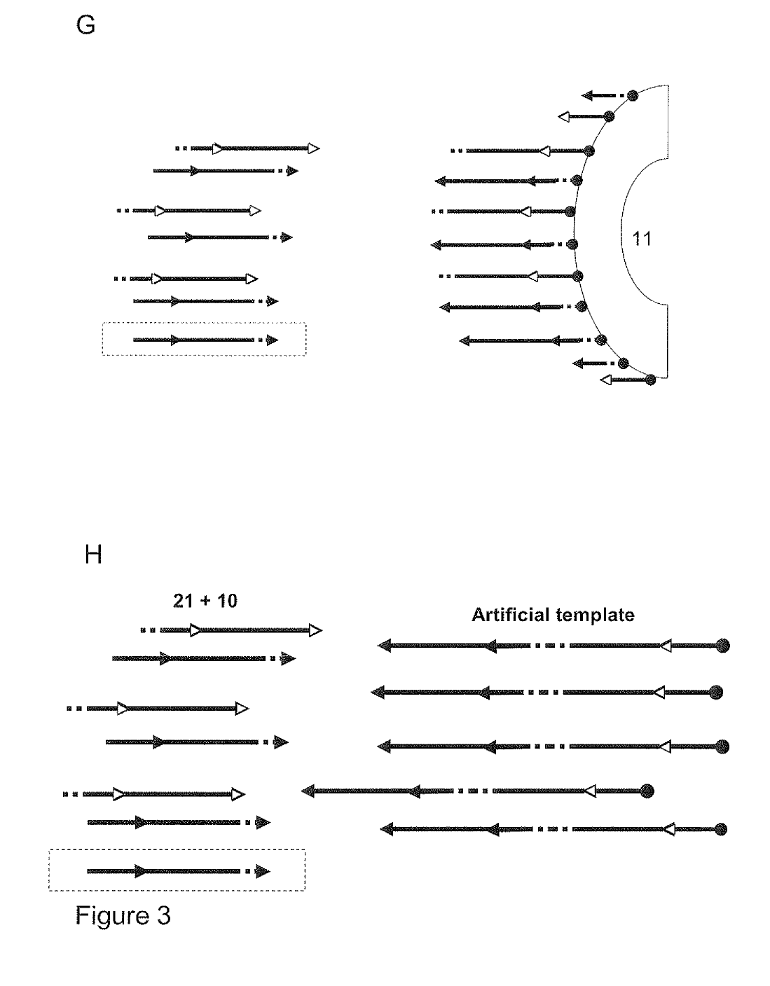

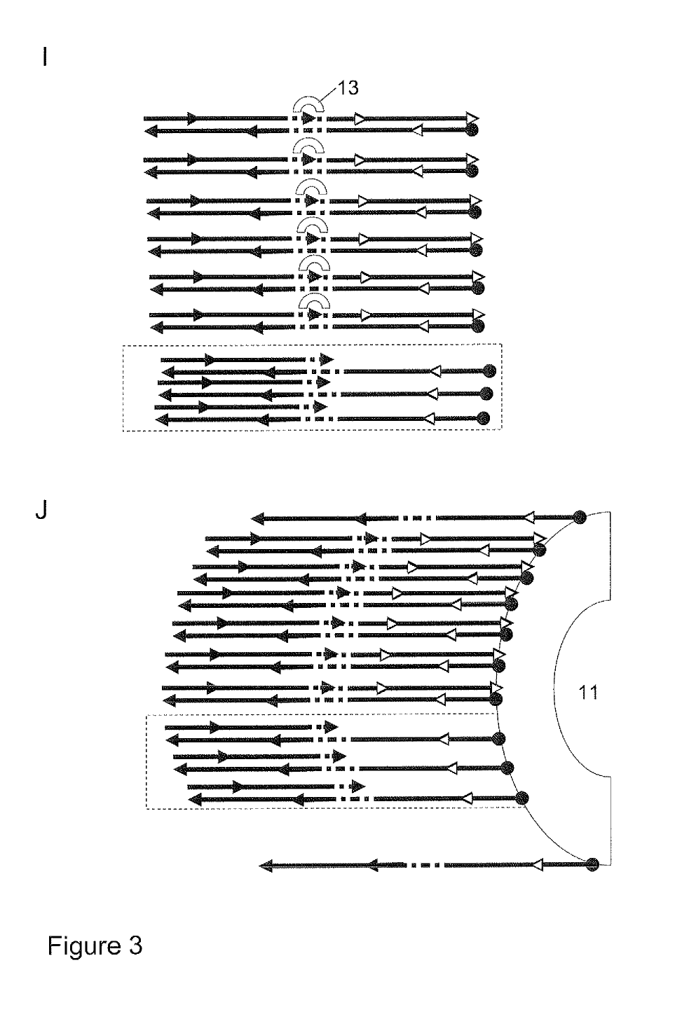

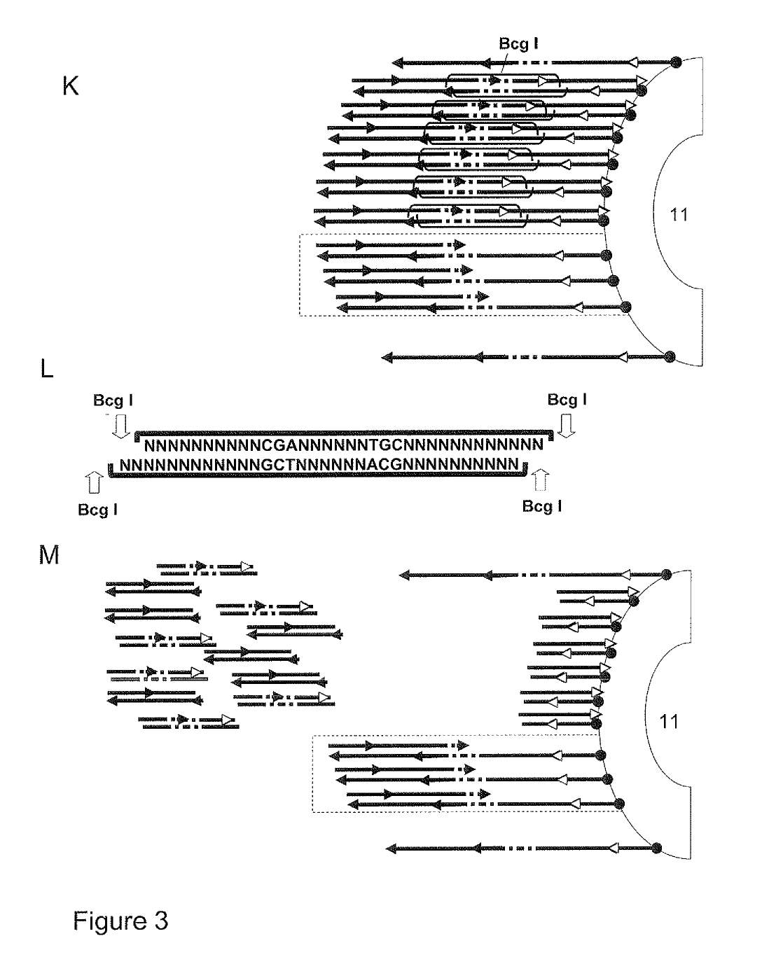

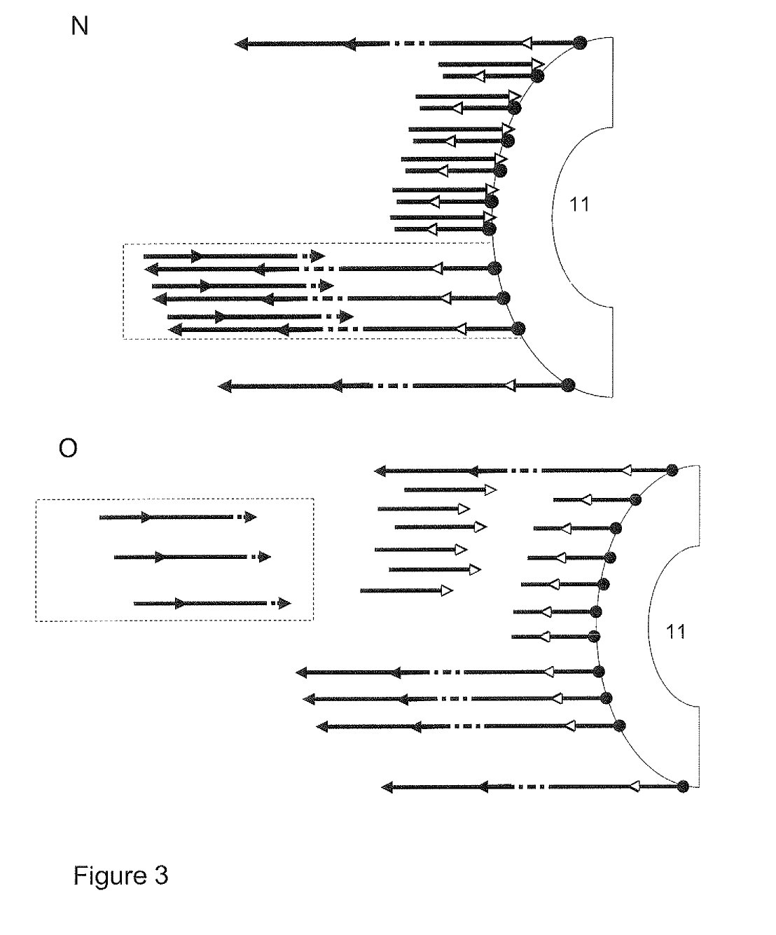

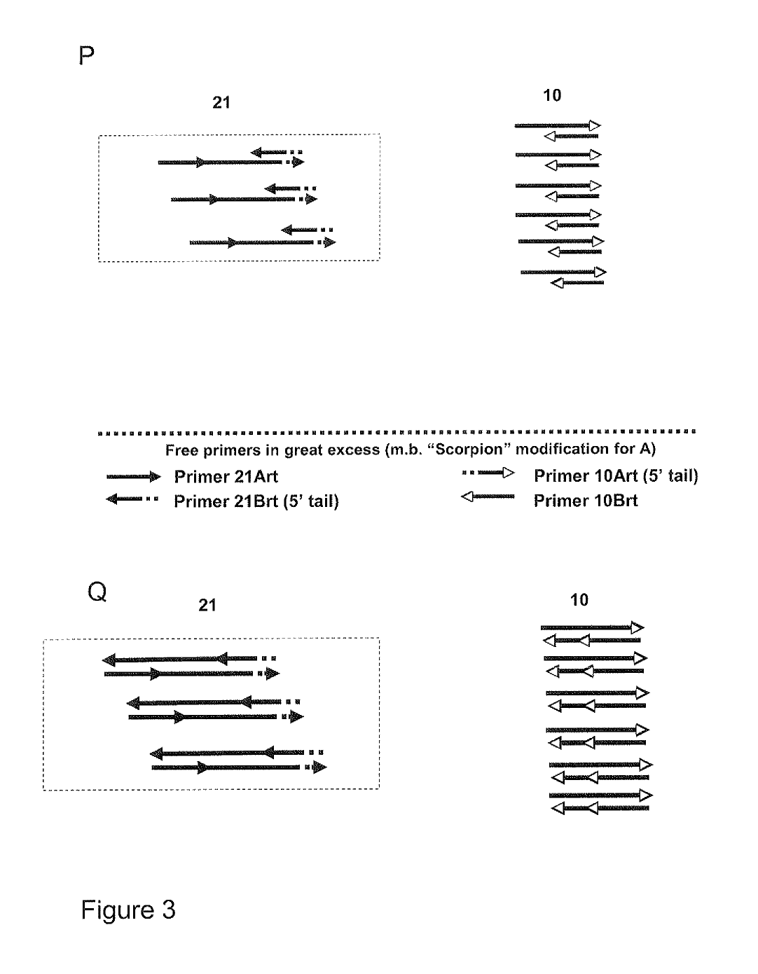

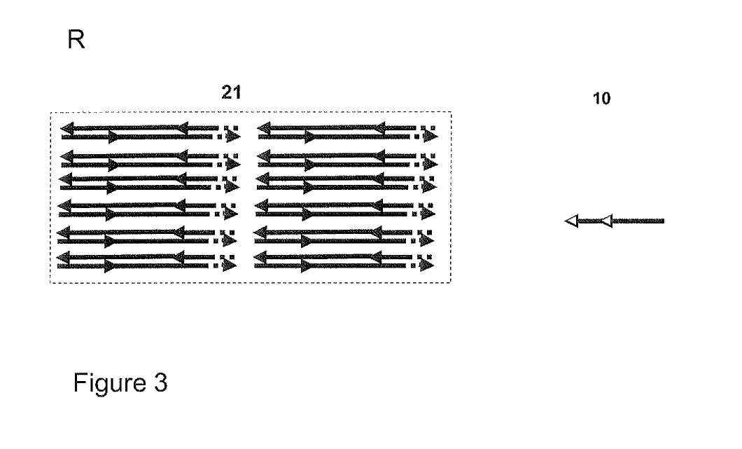

FIGS. 3A-R—show schematic diagrams illustrating the steps of the first embodiment of the invention. In particular FIGS. 3A-G—illustrate the amplification and purification of first and second sequences of nucleic acid; FIGS. H-J—illustrate the association of first and second sequences of nucleic acid; FIGS. K-N—illustrate the elimination of associated nucleic acid; and FIGS. O-R—illustrate the purification, amplification and detection of the first sequence of nucleic acid;

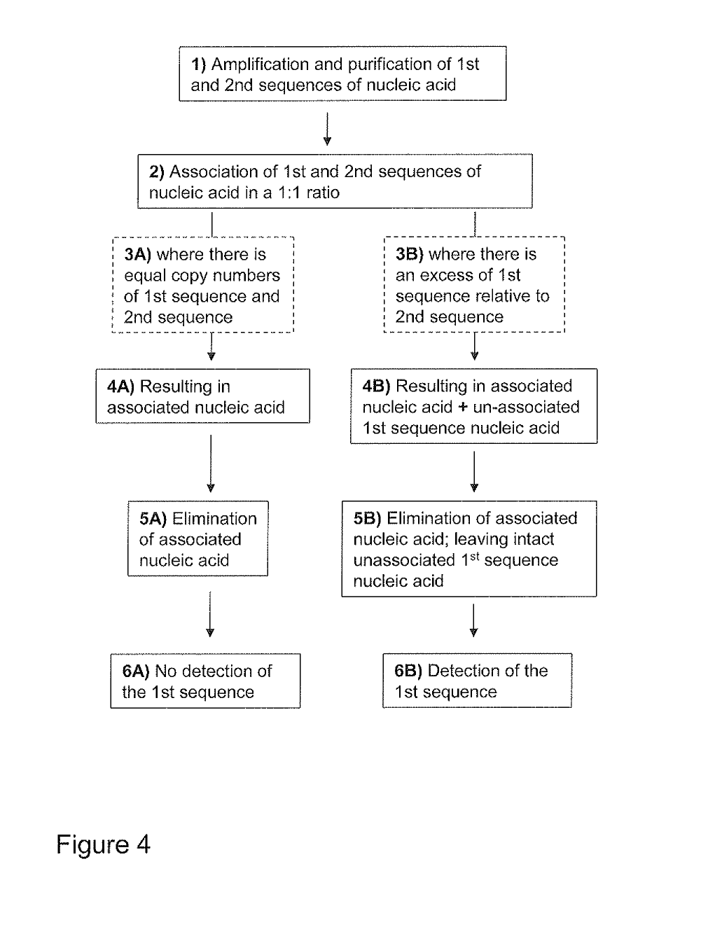

FIG. 4—illustrates general steps of a second embodiment of the invention;

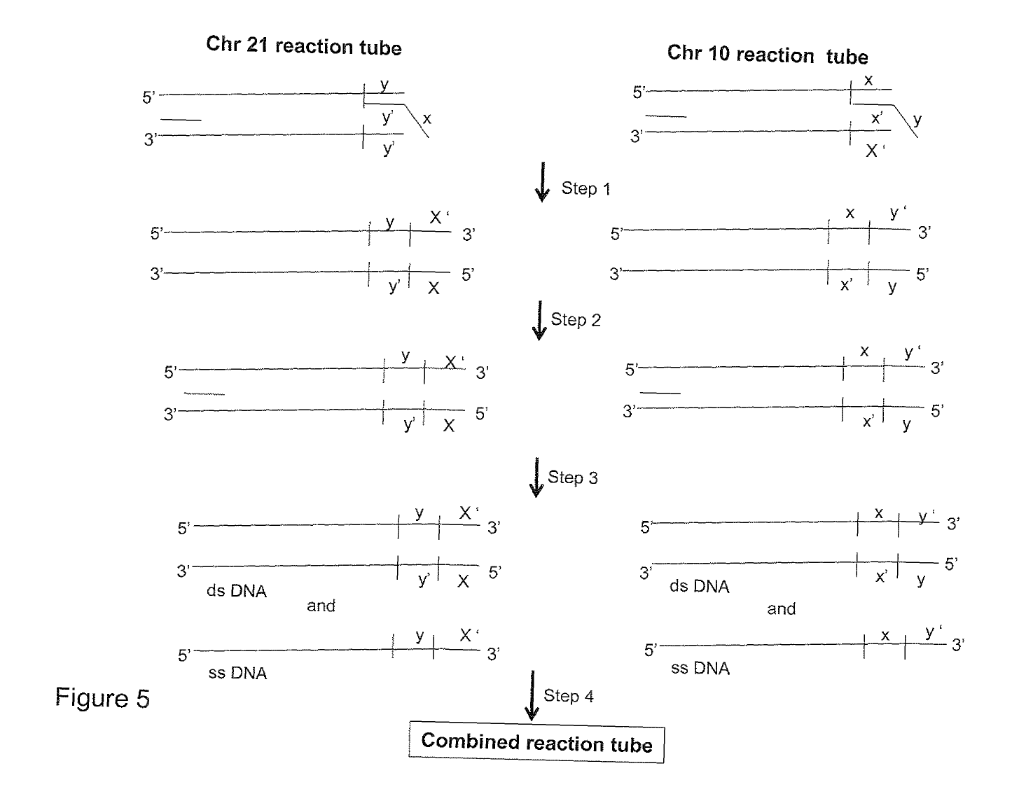

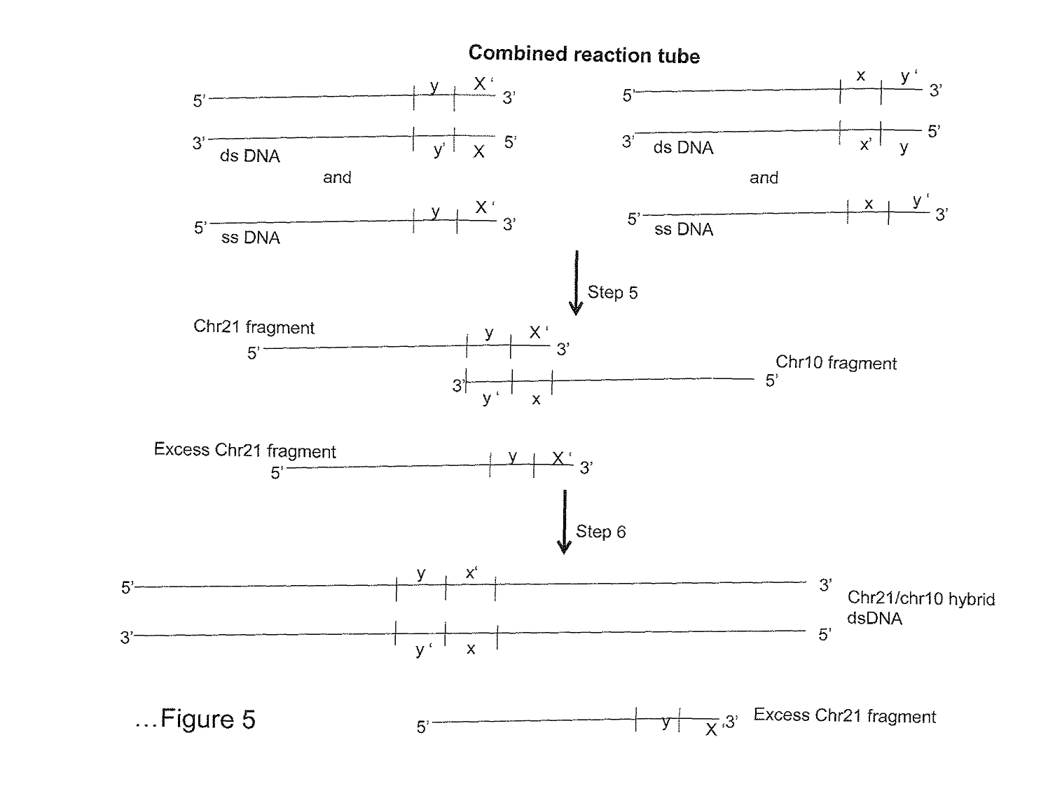

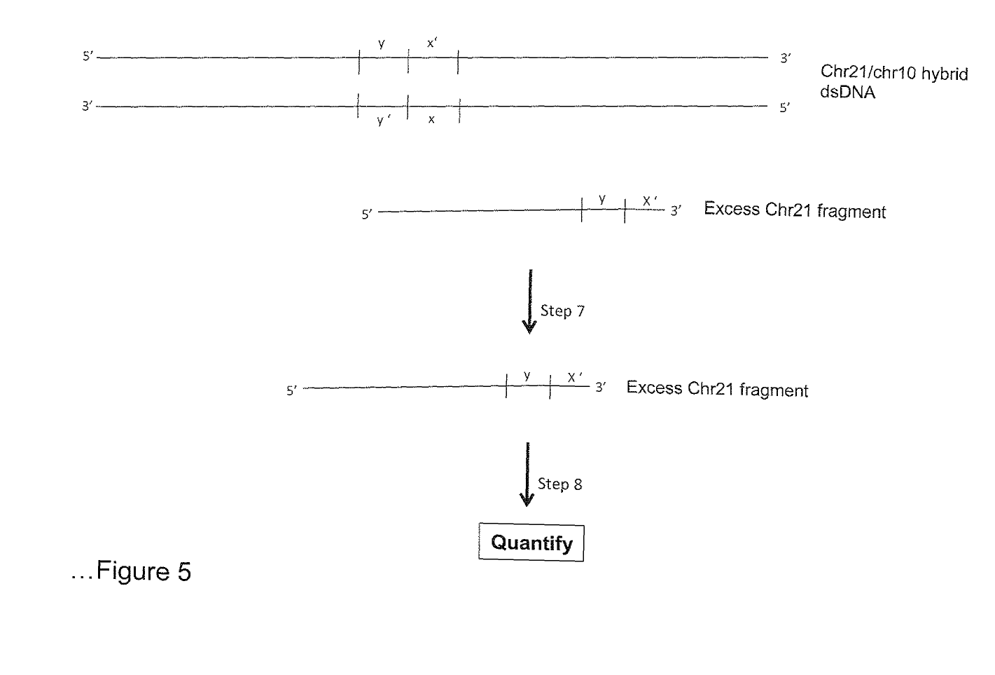

FIG. 5—is a schematic diagram showing the steps of the second embodiment of the invention;

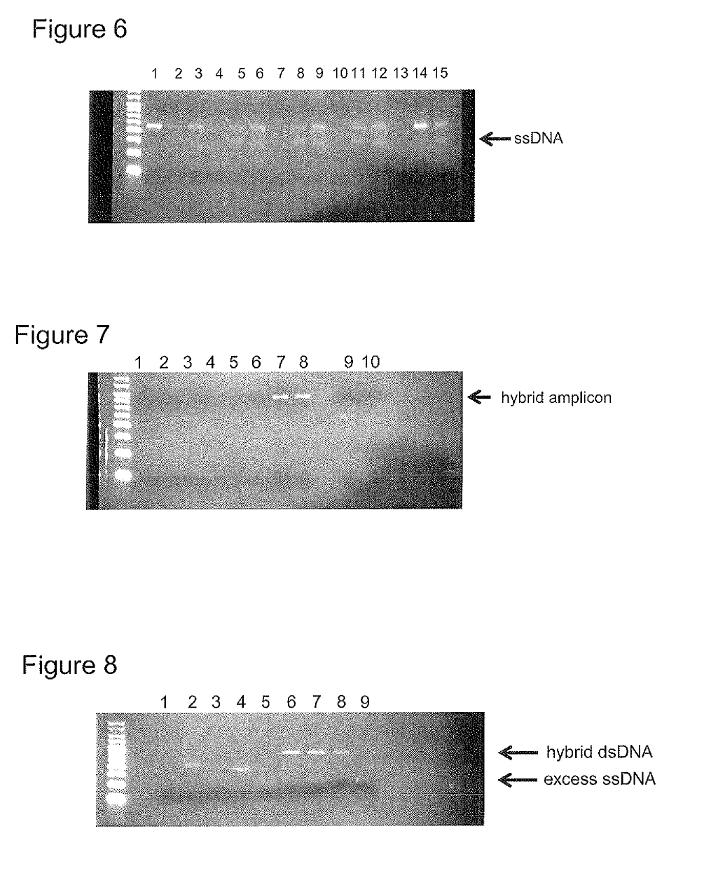

FIG. 6—shows production of single stranded DNA (ssDNA) by asymmetric PCR according to the method illustrated in FIGS. 4 and 5; lane 1 full-length dsDNA symmetric PCR; lanes 2, 5, 8, 11 and 14 using un-purified symmetric PCR and increasing Forward primer, 0.2 mM, 0.4 mM, 0.8 mM, 1.0 mM, and 1.5 mM, respectively. Lanes 3, 6, 9, 12, and 15 use the same primer concentration range, but were carried out using MSB kit purified symmetric PCRs. This figure was produced using whole male genomic DNA as the template;

FIG. 7—shows production of hybrid dsDNA of the target and reference genes according to the method illustrated in FIGS. 4 and 5; Lanes 2, 3, and 5, 6 are asymmetric PCRs of the target and reference genes, respectively. Lanes 7 and 8 are the hybrid dsDNA of the target and reference post mixing and extension. Lanes, 9 and 10 are the symmetrical PCRs of the target and reference respectively. This figure was produced using whole male genomic DNA as the template;

FIG. 8—shows restriction endonuclease elimination of the hybrid dsDNA of the target and reference genes according to the method illustrated in FIGS. 4 and 5; Lanes 2, 4, and 3, 5 are the symmetric and asymmetric PCRs of the target and reference genes respectively. Lanes 6 and 7 are bands of the hybrid/associated dsDNA of the target and reference post mixing. Lane 8 is the hydbrid/associated dsDNA post purification mock RE treatment. Lane 9 hybrid dsDNA treated with REs, hybrid/associated DNA is eliminated and excess ssDNA indicated by the bottom arrow. This figure was produced using whole-genome amplified maternal serum DNA as the template;

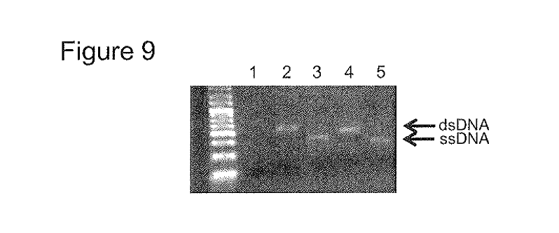

FIG. 9—shows production of ssDNA by asymmetric PCR according to the method illustrated in FIGS. 4 and 5; lanes 2 and 4 are the symmetric PCRs of the target and reference genes respectively. Lanes 3 and 5 are the asymmetrical PCRs of the target and reference genes respectively. This figure was produced using whole-genome amplified maternal serum DNA as the template;

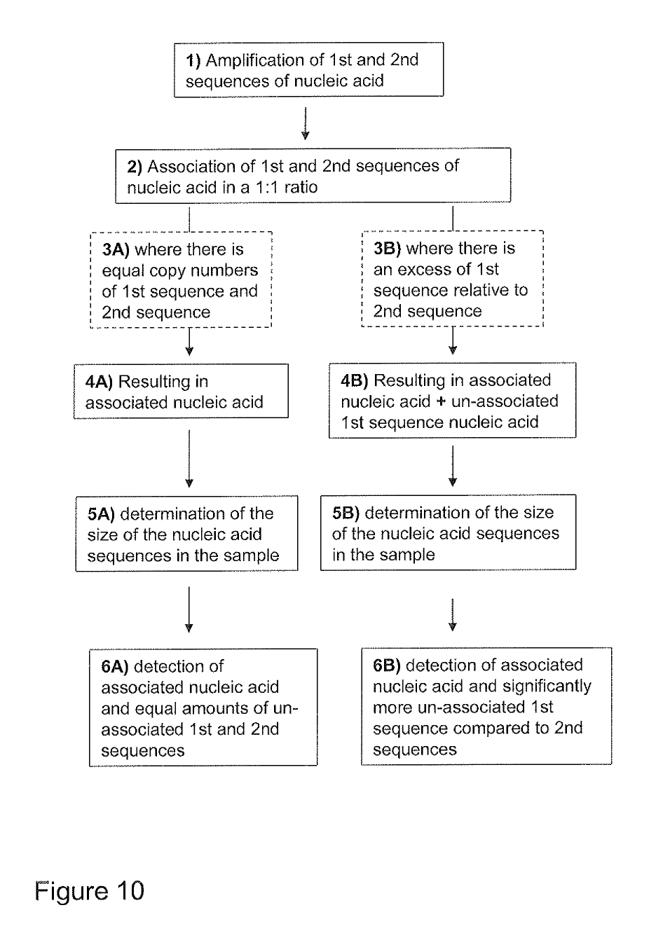

FIG. 10—illustrates general steps of a third embodiment of the invention;

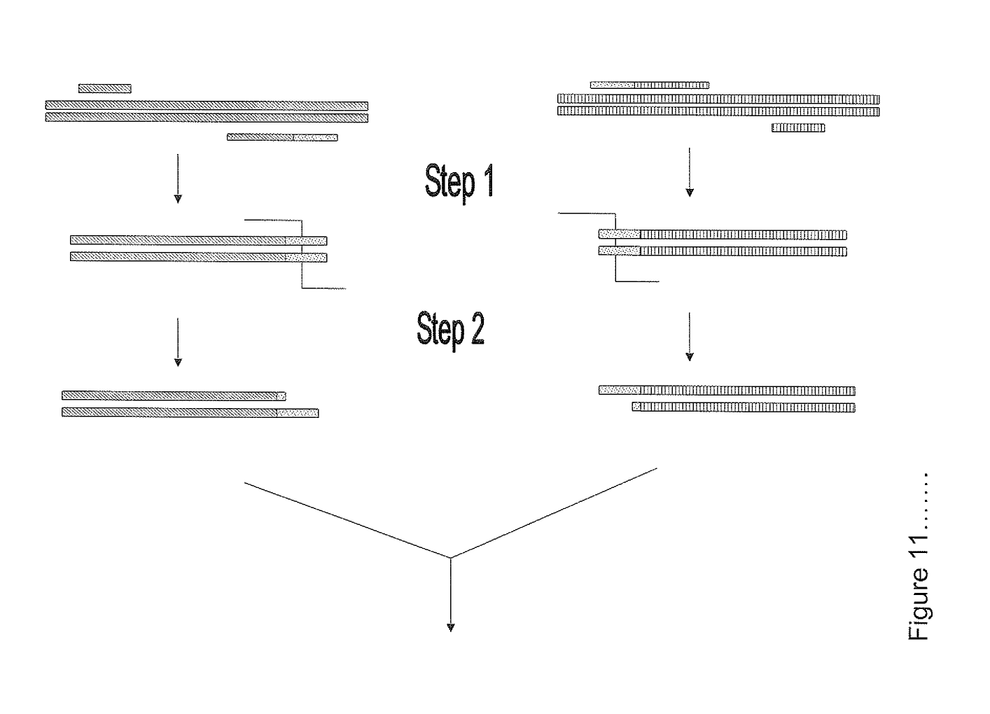

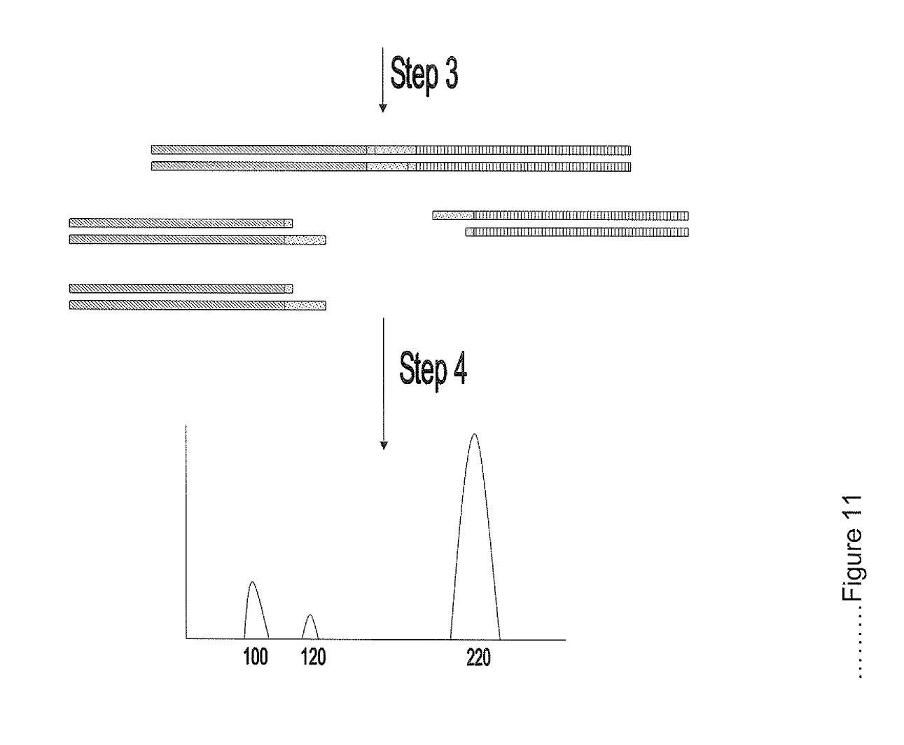

FIG. 11—is a schematic diagram showing in more details the steps of the third embodiment of the invention;

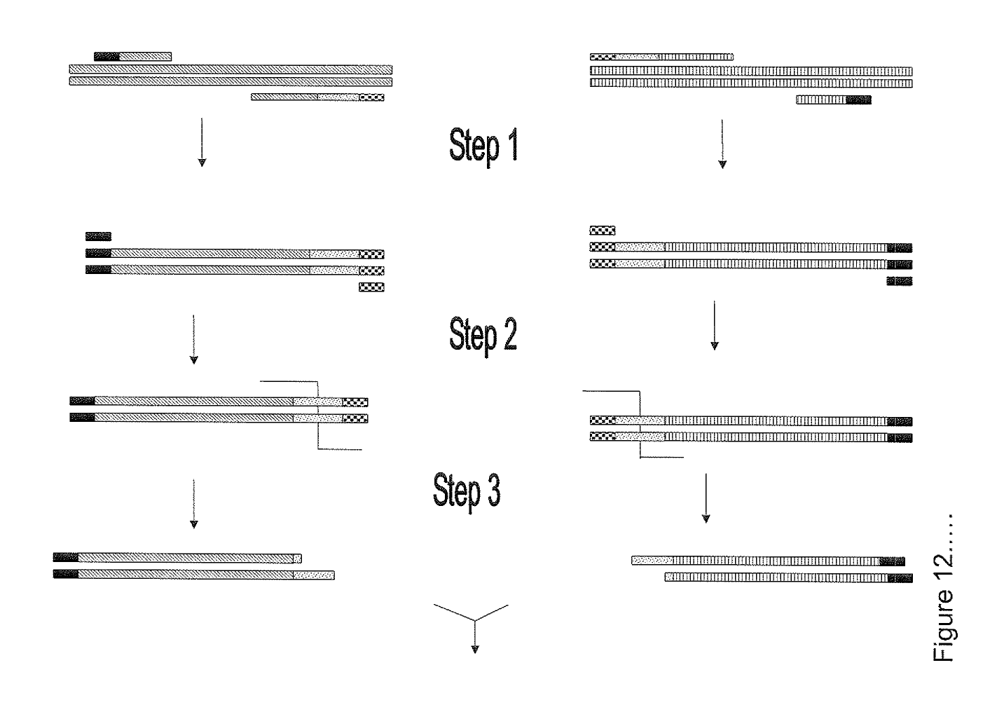

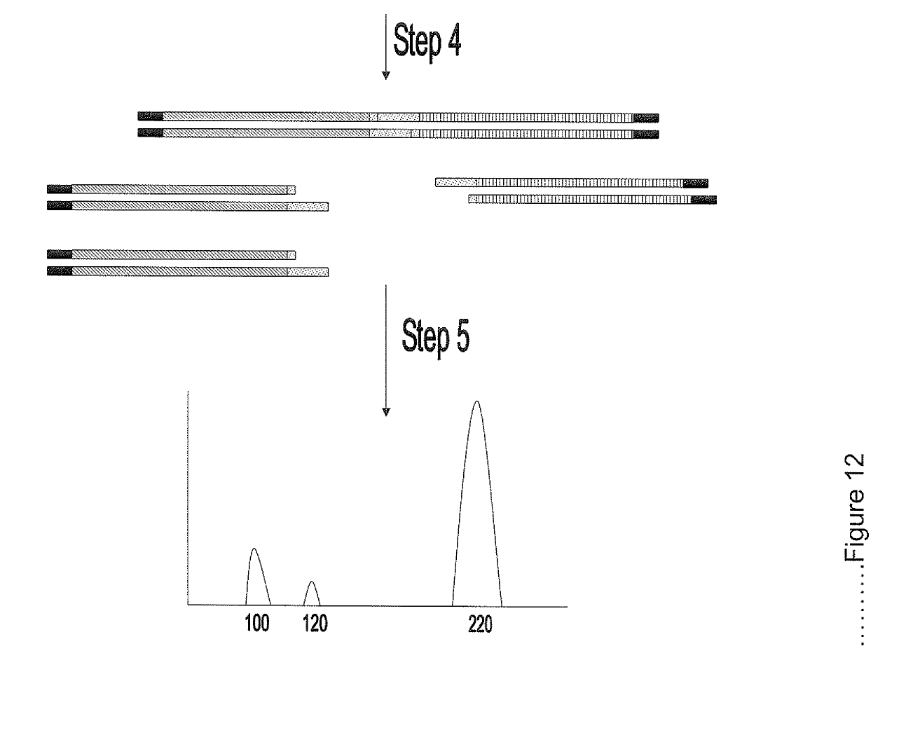

FIG. 12—is a schematic diagram showing in more details the steps of the third embodiment of the invention as depicted in FIG. 11, however in this version the initial primers also include an anchor sequence; and

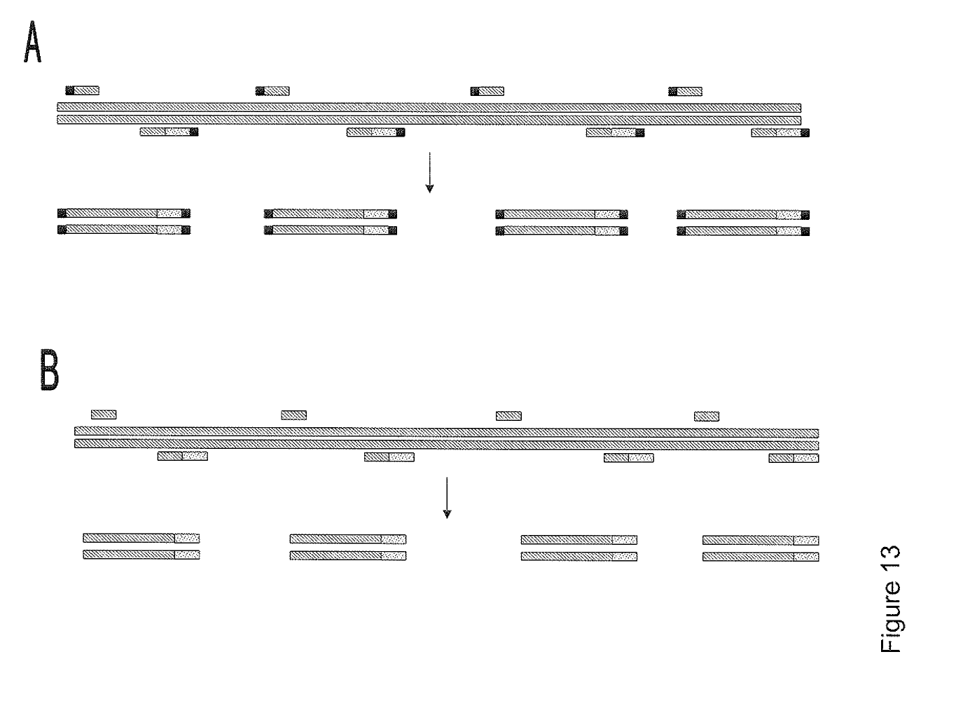

FIGS. 13A and B—illustrate the embodiments of FIGS. 12 and 11 respectively in which multiple sequences are amplified.

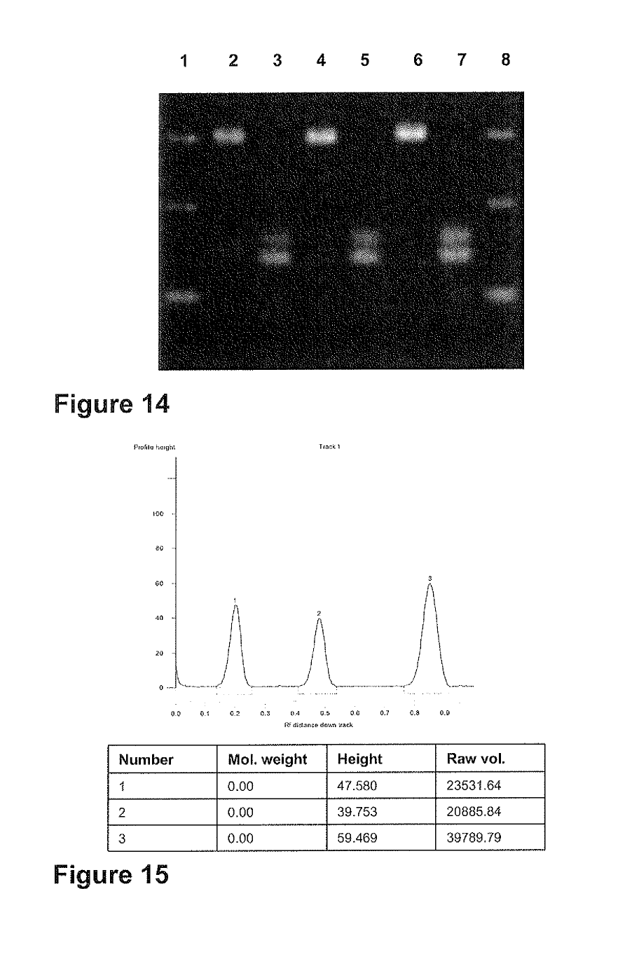

FIG. 14—shows an electrophoresis gel detailing the results of the ligation of a 146 bp Chromosome 2 fragment and a 165 bp Chromosome 21 fragment when mixed in different ratios. Various control are also included. More specifically the tracks are as follows. Track 1 and 8—a 100 bp DNA ladder 100 bp (invitrogen). Track 2—Fragments of Chromosome 2 and 21 in a 2:1 ratio with ligase, Chromosome 2 is in excess—the lower band is excess chr 2 fragment and the higher band is Chr 21 and 2 fragments ligated. Track 3—Fragments of Chromosome 2 and 21 in a 2:1 ratio without ligase, Chromosome 2 is in excess. The lower band is chr 2 fragment and the higher band is Chr 21 fragment. Track 4—Fragments of Chromosome 2 and 21 in a 1.5:1 ratio with ligase, Chromosome 2 is in excess—the lower band is excess chr 2 fragment and the higher band is Chr 21 and 2 fragments ligated. Track 5—Fragments of Chromosome 2 and 21 in a 1.5:1 ratio without ligase, Chromosome 2 is in excess. The lower band is chr 2 fragment and the higher band is Chr 21 fragment. Track 6—Fragments of Chromosome 2 and 21 in a 1:1 ratio with ligase, The only band seen is ligated Chr 21 and 2 fragments. Track 7—Fragments of Chromosome 2 and 21 in a 1:1 ratio without ligase. The lower band is chr 2 fragment and the higher band is Chr 21 fragment;

FIG. 15—illustrates the results of analysis of track 1 of the gel in FIG. 14 using a Syngene Image Analyser. The results illustrate the position of the bands of the gel (as distance down track) and the intensity of the bands (as profile height;

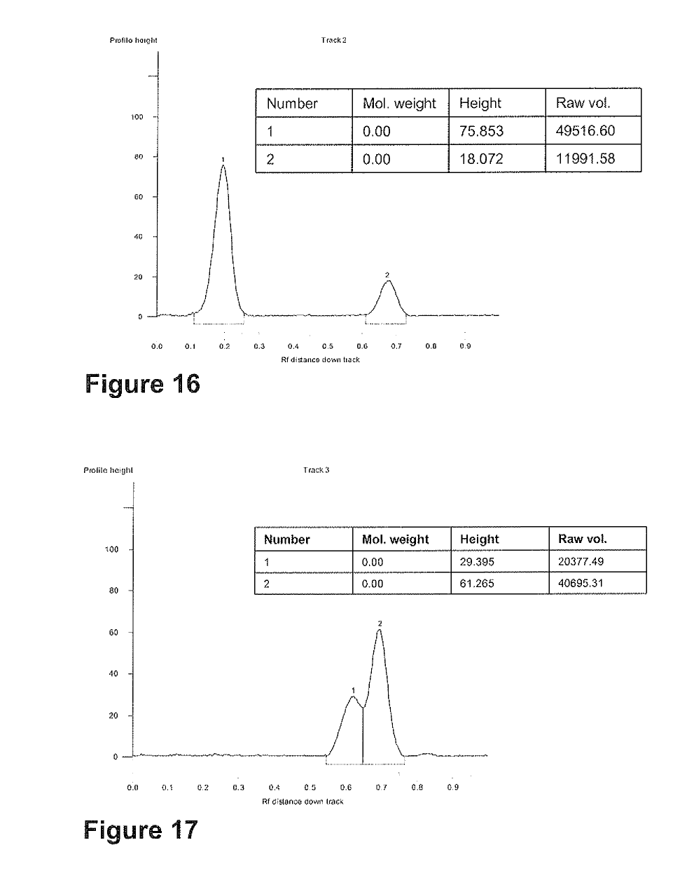

FIG. 16—is as FIG. 15 but with respect to track 2;

FIG. 17—is as FIG. 15 but with respect to track 3;

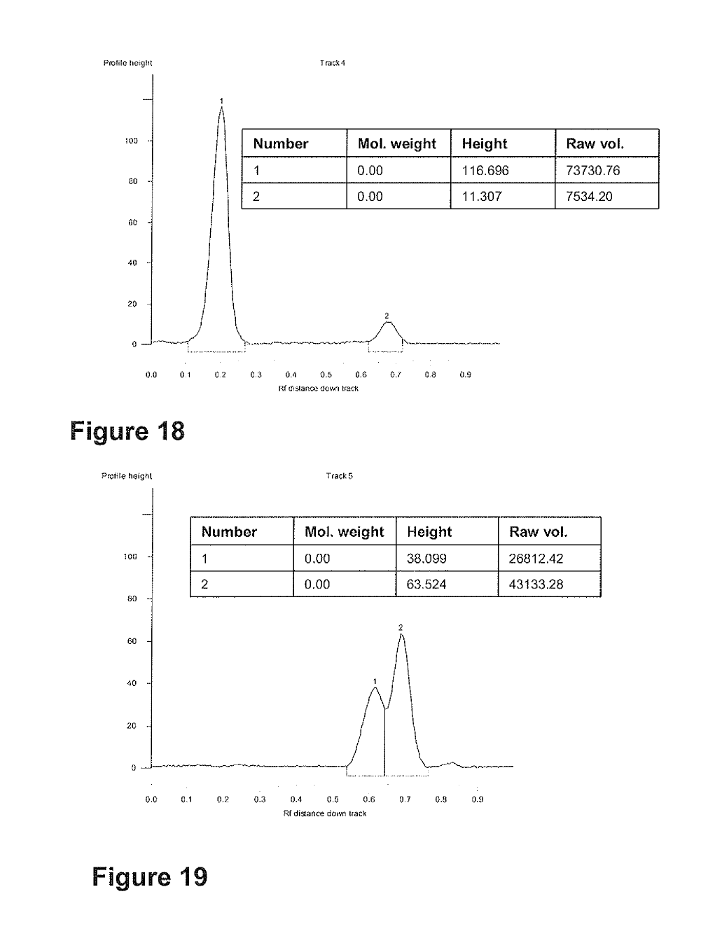

FIG. 18—is as FIG. 15 but with respect to track 4;

FIG. 19—is as FIG. 15 but with respect to track 5;

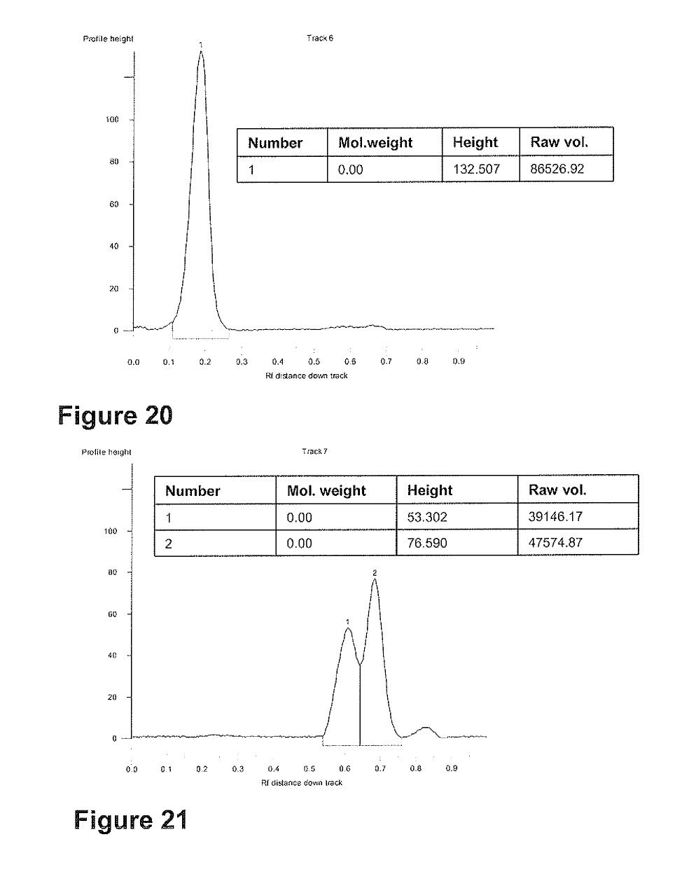

FIG. 20—is as FIG. 15 but with respect to track 6;

FIG. 21—is as FIG. 15 but with respect to track 7;

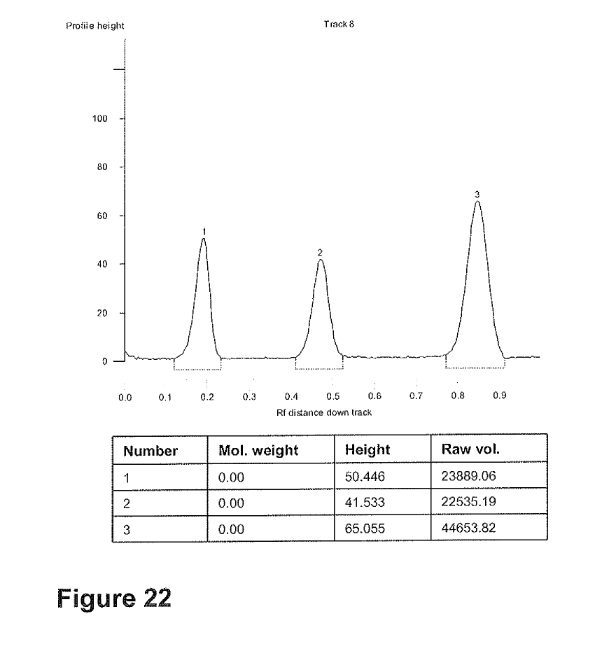

FIG. 22—is as FIG. 15 but with respect to track 8.

### EXAMPLE 1

### First Embodiment of the Invention

The following example demonstrates the use of the method of the invention for the detection of Down's Syndrome, however, the skilled person will recognise that an excess of deficiency in copy number of a any chromosome, gene, or nucleic acid sequence may be detected according to the principles of this invention.

With reference to FIGS. 2 and 3 the method of the invention can be carried out as follows.

### Step 1

Maternal blood plasma is provided from a pregnant woman, which comprises both maternal and foetal DNA (FIG. 3A). The foetus may or may not have an extra copy number of chromosome 21.

### Step 2

With particular reference to FIGS. 3B-E, DNA is extracted from a sample of the maternal blood plasma and used as the template DNA in a polymerase chain reaction (PCR). The PCR uses a pair of primers (21A and 21B) complementary to a target region of chromosome 21 and a pair of primers (10A and 10B) complementary to a target region of chromosome 10. In this example chromosome 10 is used, however, the skilled person will understand that any other suitable chromosomal region which is present in normal (duplicate) copy number can be used. The primers have the characteristics described in Table 1A.

TABLE 1A

| | |
|---|---|
| Primer 21A | complementary to a region of chromosome 21 |
| Primer 21B | complementary to a region of chromosome 21, and |
| | comprises a 5′ biotin tag and a 5' tail sequence |
| | of 5′-NNNTCG-3′ |
| Primer 10A | complementary to a region of chromosome 10, and |
| | comprises a 5' tail sequence of 5′-NNNTCG-3′ |
| Primer 10B | complementary to a region of chromosome 10, and |
| | comprises a 5′ biotin tag |

### Step 3

Once several cycles of PCR amplification have occurred, the resulting amplification product comprises multiple copies of chromosome 21 target region and multiple copies of chromosome 10 target region (FIG. 3E). These copies are in the form of double-stranded DNA, where one of the strands is biotinylated.

Where there is an excess copy number of chromosome 21 in the maternal blood plasma, the resulting PCR amplification product comprises a slight excess in the number of copies of the chromosome 21 target region (highlighted by a dashed-line box). Such a slight excess of copy number is not distinguishable using conventional quantitative methods.

### Step 4

With particular reference to FIG. 3F, the double-stranded copies of chromosome 21 and 10 target region are immobilised on beads (11) using the biotin-tag, which is present on one of the strands of the double-stranded DNA. Also some unused biotinylated primers are immobilised on the beads (11).

The beads (11) are then washed to remove any non-biotinylated DNA, genomic DNA, enzymes, dNTP's, primers and other unwanted components remaining in the PCR amplification mixture.

### Step 5

With reference to FIG. 3G, the bead (11) immobilised copies of chromosome 21 and 10 are chemically denatured. Chemical denaturing can be carried out using alkaline denaturation by adding sodium hydroxide in order to bring the pH to 12.0-12.5. This treatment releases non-biotinylated single-stranded copies of chromosome 21 and 10 target region from the beads.

### Step 6

The biotinylated strands and any unused biotinylated primers remain immobilised on the beads (11). The beads (11) are magnetically recovered and discarded to leave single-stranded copies of chromosome 21 and 10 target region (FIG. 3G).

### Step 7

With reference to FIG. 3H, an artificial DNA template is added to the single-stranded copies of chromosome 21 and 10 target region.

The artificial DNA template is a single-stranded DNA molecule comprising a 5′ portion complementary to the copies of chromosome 10 target region and a 3′ portion complementary to the copies of chromosome 21 target region. The two portions are linked and spaced apart by a restriction enzyme recognition sequence 5′-GCANNNNNNTCG-3′. This sequence is recognised by Bcg I once both the copies of chromosomes 21 and 10 target region (each having 5′ tails which are complementary to respective halves of the restriction enzyme recognition sequence) are annealed to form a double-stranded restriction enzyme recognition site.

The artificial DNA template is also biotinylated at its 5′ end.

### Step 8

With reference to FIG. 3I, the single-stranded copies of chromosome 21 and 10 target region are annealed to their respective complementary portion on the artificial DNA template, such that the copies of chromosome 21 and 10 target region are associated with each other. The annealing can be facilitated by reducing the pH, by addition of acid. Alternatively, or additionally, the annealing can be facilitated by an additional purification step prior to step 7.

The annealing forms an associated double-stranded complex having copies of chromosome 21 and 10 annealed to the artificial DNA template in equal number (i.e. a 1:1 ratio). There is a gap between the associated copies of chromosome 21 and 10 target region because the complementary portions of artificial DNA template are spaced apart by the restriction enzyme recognition sequence.

Where there are excess copies of chromosome 21 target region relative to copies of chromosome 10 target region, there is not enough copies of chromosome 10 target region to pair with, and associate with, the excess copy numbers of chromosome 21 target region in a 1:1 ratio. Thus, this results in an un-associated DNA complex where some artificial DNA template has only a copy of chromosome 21 target region annealed to the relevant complementary portion (highlighted by a dashed-line box). The chromosome 10 complementary portion remains as a single-stranded 5′ tail.

There may be some accidental annealing of copies of chromosome 10 target region to the artificial DNA template without a copy of chromosome 21 target region also annealing to the artificial DNA template. This effect should be negligible.

### Step 9

The associated copies of chromosome 21 and 10 target region are treated with a thermostable ligase (Ampligase®) (13) which fills in the gap between copies of chromosome 21 and 10 target region using polymerase activity, and ligates the copies of chromosome 21 and 10 target region together (FIG. 3J). The polymerase activity completes the Bcg 1 restriction enzyme recognition site, such that it is double-stranded.

The un-associated DNA complex, if any, has only a single copy of chromosome 21 target region annealed, thus no ligation is possible.

Any negligible amount of accidental un-associated DNA complex having only a copy of chromosome 10 target region would also not be ligated.

### Step 10

With reference to FIG. 3J, the associated and any un-associated DNA complexes are immobilised on beads (11) by binding the biotin-tag on the 5′ end of the artificial DNA template.

### Step 11

Enzymes and all unwanted unbound DNA are washed off from the beads (11) (FIG. 3J).

### Step 12

With reference to FIGS. 3K-M, restriction enzyme Bcg 1 is used to cut the bead (11) immobilised associated DNA complex twice at flanking regions of the recognition sequence (FIG. 3L) such that truncated fragments of associated DNA complex comprising copies of chromosome 21 target region and artificial DNA template are released from the beads (11) and truncated copies of associated DNA complex comprising chromosome 10 target region and artificial DNA template remain immobilised on the beads (11) (FIG. 3M).

Bcg I is a restriction enzyme which recognises the sequence of 5′-GCANNNNNNTCG-3′ in double stranded DNA. The actual cut site (arrows) is 12 base pairs down stream of this sequence (see FIG. 3L). As a consequence the cut site truncates the down stream copy of chromosome 10 or 21 target region.

Any un-associated DNA complex is not cut because the restriction enzyme recognition sequence has not been completed and remains single-stranded. Thus, un-associated DNA complex remains immobilised to the beads (11) (highlighted by a dashed-line box).

### Step 13

The beads (11) are washed in an appropriate buffer to remove unbound DNA and enzymes (FIG. 3M).

### Step 14

With reference to FIGS. 3N and 3O, the immobilised double-stranded copies of any un-associated DNA complex and truncated associated DNA complex are chemically denatured by increasing the pH (e.g. by adding sodium hydroxide in order to bring the pH to 12.0-12.5, as in step 5). This releases truncated copies of chromosome 10 target region, and where there is un-associated DNA complex, it releases intact copies of chromosome 21 target region (highlighted by a dashed-line box) (FIG. 3O).

Negligible amounts of accidental intact copies of single-stranded chromosome 10 target region may also be released.

The biotinylated fragments of artificial DNA template remain bound to the beads (11).

### Step 15

With reference to FIG. 3O, the beads (11) are magnetically collected and discarded to leave behind any single-stranded intact copies of chromosome 21 target region (highlighted by a dashed-line box) and single-stranded truncated copies of chromosome 10 target region.

There may also be a negligible amount of intact single-stranded copies of chromosome 10 target region which have been accidently preserved.

### Step 16

With reference to FIGS. 3P-R, the truncated copies of chromosome 10 target region and any intact copies of chromosome 21 target region are amplified in a real-time PCR amplification.

The real-time PCR amplification uses the primers having the characteristics described in Table 2.

TABLE 2

| | |
|---|---|
| Primer 21Art | complementary to the same region of chromosome 21 as |
| | Primer 21A |
| Primer 21Brt | complementary to the same region of chromosome 21 as |
| | Primer 21B, and comprises a 5′ tail |
| Primer 10Art | complementary to the same region of chromosome 10 as |
| | Primer 10A, and comprises a 5′ tail |
| Primer 10Brt | complementary to the same region of chromosome 10 as |
| | Primer 10B |

The truncated copy of chromosome 10 target region can only amplify linearly due to only one of the primers having a binding site. The other primer binding site has been removed by the restriction enzyme cut made in step 12 to form the truncated copy.

Any intact copy of chromosome 21 target region is amplified exponentially (highlighted by a dashed-line box).

Any accidentally preserved copies of chromosome 10 target region would also be amplified exponentially, but the final amount would only be detectable at a low level.

### Step 17

The amplification products are quantitatively detected throughout the real-time PCR amplification using either Taqman® probes or Scorpion® primers.

In a scenario where there were excess copies of chromosome 21 target region then this would be detected in abundance relative to any other amplification product (See FIG. 3R). Negligible amounts of truncated copies of chromosome 10 may also be detected.

Copies of chromosome 10 target region may be detectable, but only at a low level.

In a scenario where there was equal copy numbers of chromosome 21 and 10 target region, then there would be no, or only a low level of chromosome 21 or 10 target sequence detectable, or truncated copies thereof.

### Step 18

If an abundance of copies of chromosome 21 target region is detected, a diagnosis of Down's Syndrome is made.

If no abundance of copies of chromosome 21 target region is detected, then no diagnosis of Down's Syndrome is made.

The skilled person will recognise that in alternative embodiments, the above steps can be performed in a different order where appropriate, as readily determined by a skilled person. For example, denaturing the double-stranded DNA may be carried out before or after immobilising the strands onto the beads using the biotin-tag.

### Other Examples

The method of the invention can be used in an assay for detecting quantitative differences between sequences representing HER2/neu (c-erbB-2) gene (located at 17q21.1) and a control gene such as a housekeeping gene encoding glyceraldehyde-3-phosphate dehydrogenase (GAPDH) as a somatic cell application. The first (HER2) primer pair may comprise a forward primer of the sequence 5′-GTGAGGGACACAGGCAAAGT-3′ and a reverse primer of the sequence 5′-TGCAAGTGCAATACCTGCTC-3′. The second (GAPDH) primer pair may comprise a forward primer of the sequence 5′-CTCCCACCTTTCTCATCCAA-3′ and a reverse primer of the sequence 5′-GTCTGCAAAAGGAGTGAGGC-3′.

As another example, the method of the invention can be used in an assay for detecting chromosome 21 trisomy by comparing Down Syndrome Candidate Region 1 (DSCR1) also known as Regulator of Calcineurin 1 (RCAN1) gene located on chromosome 21 (21q22.12) with the same GAPDH gene located on chromosome 12 (12p13). The first (DSCR1) primer pair may comprise a forward primer of the sequence 5′-AGTCCTGGGACCAGAAGGTT-3′ and a reverse primer of the sequence 5′-GCAGAGTAAAACCAGCAGGC-3′. The second (GAPDH) primer pair may comprise a forward primer of the sequence 5′-CTCCCACCTTTCTCATCCAA-3′ and a reverse primer of the sequence 5′-GTCTGCAAAAGGAGTGAGGC-3′.

### EXAMPLE 2

### Second Embodiment of the Invention

The following example demonstrates the use of the method of the invention for the detection of Down's Syndrome, however, the skilled person will recognise that an excess or deficiency in copy number of a any chromosome, gene, or nucleic acid sequence may be detected according to the principles of this invention.

### Method Summary

With reference to FIG. 5, a summary of the method is as follows.

### Step 1—Symmetric PCR Step

PCR with a forward and a tailed primer in order to separately amplify a target sequence of chromosome 21 and a reference sequence of chromosome 10.

Chromosome 21 forward primer is complementary to the antisense strand of the target sequence—Chr21

Chromosome 21 Reverse (tailed) primer comprises a portion (y′), which is complementary to region y of the sense strand of the target sequence—Chr21. Also comprises a portion (x), which is complementary to region x′ of the antisense strand of the reference sequence—Chr10.

Chromosome 10 forward primer is complementary to the antisense strand of the target sequence—Chr10

Chromosome 10 reverse (tailed) primer comprises a portion (x′), which is complementary to region x of the sense strand of the reference sequence—Chr10. Also comprises a portion (y), which is complementary to region y′ of the antisense strand of the target sequence—Chr21.

### Step 2

Purify the PCR products to remove forward and reverse (tailed) primers then use a small amount of standard PCR products for asymmetric PCR.

### Step 3—Asymmetric PCR Step

Use forward primers for asymmetric PCR.

### Step 4—Pairing/Association PCR Step

Mix amplification products from chromosome 21 and chromosome 10 reaction tubes immediately after asymmetric PCR step without purification.

### Step 5—Pairing/Association PCR Step

After mixing amplification products, put through a heat, anneal and extend cycle to extend single stranded DNA into double stranded DNA

### Step 6—Pairing/Association PCR Step

Partially double stranded sequence is extended to form a Chr21/Chr10 hybrid dsDNA (double stranded associated nucleic acid complex).

### Step 7—Restriction Endonuclease Elimination of Paired/Associated DNA

Eliminate all double stranded DNA using a double stranded DNA specific nuclease.

### Step 8

Quantify DNA by Taqman® RT-PCR amplification. The detection of an amplification product similar in length to the target sequence or reference sequence indicates a difference in copy number, which can be used for a diagnosis.

### More Detailed Method

With reference to FIG. 5, the method comprises the following steps:

### Step 1—Symmetric PCR Step

30 cycles of standard symmetric PCR with a forward and a tailed primer in order to separately amplify a target sequence of chromosome 21 and a reference sequence of chromosome 10.

### Primer Details

The primers employed in the PCR step are as follows:
Target Sequence (from Chromosome 21)

Forward primer sequence 1:
5′ CAGCCAAAGACAGAACTTAACCTC 3′
Forward primer sequence 2:
5′ CAGCCAAAGACAGAACTTAACCTC 3′

Complementary to the antisense strand of the target sequence—Chr21

Reverse primer sequence 1:
5′ GAGTATTGGTCCTGGGCTTCCGGGCTCCTAGCAACCGATTG 3′
Reverse primer sequence 2:
5′CTGGTTTGGGCTTGCCTCGGGGCTCCTAGCAACCGATTG 3′

Comprises a portion (y′), which is complementary to region y of the sense strand of the target sequence—Chr21. Also comprises a portion (x), which is complementary to region x′ of the antisense strand of the reference sequence—Chr10.

Reference/Control Sequence (from Chromosome 10)

Forward primer sequence 1:
5′ GGCAGAGGGTTCTTTGCTCTAG 3′
Forward primer sequence 2:
5′GCATGACTGTTGACCTTAAGATCC 3′

Complementary to the antisense strand of the target sequence—Chr10

Reverse primer sequence 1:
5′ CAATCGGTTGCTAGGAGCCCGGAAGCCCAGGACCAATACTC 3′
Reverse primer sequence 2:
5′CAATCGGTTGCTAGGAGCCCCGAGGCAAGCCCAAACCAG 3′

Comprises a portion (x′), which is complementary to region x of the sense strand of the reference sequence—Chr10. Also comprises a portion (y), which is complementary to region y′ of the antisense strand of the target sequence—Chr21.

Portion x = GAGTATTGGTCCTGGGCTTCC
Portion x′ = GGAAGCCCAGGACCAATACTC
Portion y = CAATCGGTTGCTAGGAGCCC
Portion y′ = GGGCTCCTAGCAACCGATTG

### PCR Conditions

A whole genome amplification of the maternal serum DNA can be carried out prior to the two-step PCR strategy. The data presented for maternal serum was subjected to whole genome amplification before the two-step PCR strategy. A two-step PCR strategy is employed for the amplification of the desired target and reference gene regions, if asymmetric PCR is used for the production of single stranded DNA (ssDNA).

PCR setup of X4 quadruplicate reactions: for amplification of target or reference sequences (Chr21 or Chr10 sequences).

| | | | For X4 mix |
|---|---|---|---|
| cff DNA maternal blood (ng) | 4.0 | μl | |
| Nuclease free H₂O | 9.66 | μl | 38.64 |
| 5X Kapa Hifi Hot start GC buffer | 4.0 | μl | 16 |
| 25 mM dNTPs (300 μM final) | 0.24 | μl | 0.96 |
| 5 μM Forward primer | 0.8 | μl | 3.2 (X1) |
| reference (chr 10) | | | |
| 5 μM Reverse primer | 0.8 | μl | 3.2(X1) |
| reference (chr 10) | | | |
| Kapa Hifi Hot start DNA polymerase | 0.5 | μl | 2.0 |
| Total reaction volume | 20 | μl | |

Add 16 ul of mix per PCR tube
   PCR cycle: 95° C. for 5 minutes then, 98° C. 20 sec, 60° C. 20 sec, 72° C. 30 sec, 30 cycles, then 72° C. for 5 minutes.

After PCR, purify the individual PCRs using Invitek's MSB PCR purification kit. Proceed to asymmetric step.

### Step 2

Purify the PCR products to remove forward and tailed primers then use a small amount of standard PCR products for asymmetric PCR.

### Step 3—Asymmetric PCR Step

Add forward primers for 30 cycles of asymmetric PCR.

| Asymmetric PCR setup: | | | For X5 mix |
|---|---|---|---|
| 1st PCR (for chr21 or 10 above) | 4.0 | μl | |
| Nuclease free H₂O | 10.94 | μl | 54.7 |
| 5X Kapa Hifi Hot start GC buffer | 4.0 | μl | 20 |
| 25 mM dNTPs (300 μM final) | 0.24 | μl | 1.2 |
| 25 μM Forward primer | 0.32 | μl | 1.6(X1) |
| reference (chr 21) | | | |
| Kapa Hifi Hot start DNA pol | 0.5 | μl | 2.5 |
| Total reaction volume | 20 | μl | |

Add 16 ul of mix per PCR tube
PCR cycle: 95° C. for 5 minutes then, 98° C. 20 sec, 60° C. 20 sec, 72° C. 30 sec, 30 cycles.

### Step 4—Pairing/Association PCR Step

Mix amplification products from chromosome 21 and chromosome 10 reaction tubes immediately after asymmetric PCR step without purification.

### Step 5—Pairing/Association PCR Step

After mixing amplification products, heat, anneal and elongate by cycling as follows: 95° C. for 5 minutes then, 98° C. 20 sec, 68° C. 20 sec, 72° C. 60 sec, 1 cycle.

Note: If the individual asymmetric PCRs are purified using Invitek's MSB kit before mixing, add dNTPs to a final concentration of 200 μM, and 0.5 units of Kapa Hifi DNA polymerase, then cycle as follows: 95° C. for 5 minutes then, 98° C. 20 sec, 68° C. 20 sec, 72° C. 60 sec, 1 cycle.

### Step 6—Pairing/Association PCR Step

Partially double stranded sequence is extended to form a Chr21/Chr10 hybrid dsDNA (double stranded associated nucleic acid complex).

### Step 7—Restriction Endonuclease Elimination of Paired/Associated DNA

Eliminate all double stranded DNA using restriction enzymes, a double stranded DNA specific nuclease or cross-linking.

For example, Mitomycin C forms a cross-link in the minor groove of DNA, between two guanines at their two amino groups, thus preventing the melting of the two strands when heated during PCR, preventing the amplification of the strands and effectively eliminating the double stranded DNA.

The following protocol is for the use of restriction endonuclease;

| | |
|---|---|
| 10X RE buffer | 3 μl |
| 10X BSA (optional) | 3 μl |
| dH2O | 4 μl |
| RE (5 units/μl) | 1 μl |
| MSB kit purified mixed PCR | 19 μl |

Incubate reaction mix at 37° C. for 10 minutes. Stop reaction by adding 1 μl of 0.5M EDTA. The short incubation time is due to the small quantities of DNA being handled.

Use of restriction endonucleases is one possible way to eliminate the associated dsDNA and so quantify excess ssDNA. However, in other embodiments cross-linking, and the use of a double strand specific nuclease may also be employed.

### Step 8

Quantify by Taqman® RT-PCR amplification, as follows.
Materials: Nuclease free H₂O, 10× Amplitaq Gold PCR buffer, 25 mM MgCl_{2, 25} mM dNTPs, 5 μM forward and reverse primers, 10 μM probe and Amplitaq Gold DNA polymerase (heat activated). Thaw all on ice.
Note: Carry out all procedures on ice, and ensure that all probes are wrapped up in aluminium foil to minimise exposure to light to avoid photobleaching.
PCR setup:

| PCR setup: | X1 | |
|---|---|---|
| Diluted excess ssDNA | 4.0 | μl |
| Nuclease free H₂O | 8.5 | μl |
| 10X Amplitaq Gold buffer | 2.0 | μl |
| 25 mM MgCl₂ | 3.2 | μl |
| 25 mM dNTPs | 0.16 | μl |
| 5 μM Forward primer | 0.8 | μl |
| 5 μM Reverse primer | 0.8 | μl |
| 10 μM Probe | 0.4 | μl |
| Amplitaq Gold LD DNA polymerase | 0.16 | μl |
| Total reaction volume | 20 | μl |

PCR cycle: 95° C. for 10 minutes then 50 cycles of 95° C. for 15 seconds followed by 60° C. for 45 seconds on the Rotor Gene 6000 (Corbett).

### Alternative Embodiments

Although in this embodiment the use of asymmetric PCR for the production of ssDNA is specified, in another embodiment it is also possible to use biotinylated or phosphorylated reverse primers, using streptavidin magnetic beads to purify one strand away from the other strand or lambda exonuclease to digest away a strand, leaving the other strand for the target and reference pairing/association step.

The examples herein are directed to an embodiment for prenatal diagnostics. In an alternative embodiment, this method may be applied to other uses where accurate copy number quantifications are important e.g. in the detection of cancer.

### EXAMPLE 3

### Third Embodiment of the Invention

The following example demonstrates the use of the method of the invention for the detection of Down's Syndrome, however, the skilled person will recognise that an excess or deficiency in copy number of any chromosome, gene or nucleic acid sequence may be detected according to the principles of the invention.

Method Summary with Reference to FIG. 11

With reference to FIG. 11, a summary of the method is as follows.

In the figures the cross hatched areas represent Chromosome 21 and the area hatched with vertical lines represents a reference Chromosome 2. Areas with a dotted fill represent all or part of a non-palindromic restricting enzyme sequence.

### Step 1—Symmetric PCR Step

Symmetric PCR is performed on a target sequence in Chromosome 21 and Chromosome 2.

With respect to Chromosome 21 a forward primer complementary to the chromosome is used together with a tailed reverse primer which includes a non-palindromic cut site restriction enzyme recognition sequence which becomes added to the amplified sequence of Chromosome 21. This amplified sequence is also referred to as the first nucleic acid sequence.

With respect to Chromosome 2 a reverse primer complementary to the chromosome is used together with a tailed forward primer which includes a non-palindromic cut site restriction enzyme recognition sequence which becomes added to the amplified sequence of Chromosome 2. This amplified sequence is also referred to as the second nucleic acid sequence.

The sequence included for the restriction enzyme may be sequence recognised by the enzyme BstXI.

The primers which do not include the restriction enzyme cut site may be labelled with a flourophore, such as FAM.

The result of each PCR reaction preferably produces a different sized amplification product. For the purposes of this example, the amplification involving Chromosome 21 produces an amplification product (first nucleic acid sequence) of 100 bp and the amplification product from Chromosome 2 (second nucleic acid sequence) is 120 bp.

### Step 2—Restriction Enzyme Digestion

The amplified first and second sequences are then cut with a restriction enzyme, in this case BstXI, which recognises the introduced non-palindromic restriction enzyme recognition sequence. All the amplified Chromosome 21 sequences, or first nucleic acid sequences, will have an overhanging end complementary of the same sequence. Similarly, all the amplified Chromosome 2 sequences, or first nucleic acid sequences, will have an overhanging end of the same sequence. Due to the non-palindromic nature of the restriction enzyme cut site the overhanging end on the amplified Chromosome 21 sequences will be different to the overhanging end on the amplified Chromosome 2 sequences. The different sequences are however complementary.

Purification of the amplified nucleic acids may be performed before and/or after cleavage with the restriction enzyme. In particular, purification may be performed after cleavage with the restriction enzyme to remove any small fragments produced by the cleavage.

### Step 3—Ligation and Subtractive Hybridisation

A ligation reaction is now performed to anneal the cleaved amplified Chromosome 21 (first nucleic acid sequence) and the cleaved amplified Chromosome 2 sequence (second nucleic acid sequence) in a 1:1 ratio.

There are now essentially just three sequences in a reaction tube: firstly unligated but amplified Chromosome 21 sequence (first nucleic acid sequence) which is 100 bp; secondly unligated but amplified Chromosome 2 sequence (second nucleic acid sequence) which is 120 bp; and finally ligated Chromosome 21 and 2 sequences which is 220 bp. The new ligated sequence can then be subtracted from Chromosome 21 and 10 sequences by using size discrimination. This may be achieved by any suitable method, for example gel electrophoresis or if a fluorophore has been incorporated a fragment analyser may be used to discriminate the different fragments in the reaction.

### Step 4—Determination of Fragments Present Using a Fragment Analyser

If a fragment analyser is used the ratio of the area of the peak generated by the sample chromosome (in this case Chromosome 21) and the reference chromosome (in this case Chromosome 2) sequences can be used to test normal and abnormal patients. If the ratio of Chr sample/Chr reference >1 theoretically the patient has more sample chromosome in the reaction tube suggesting an aneuploidy of the patient—in this case an increase in the number of copies of chromosome 21, hence allowing a diagnosis of Down's Syndrome. If Chr sample/Chr reference <1 theoretically the patient has less sample chromosome in the reaction tube again suggesting an aneuploidy of the patient and if Chr sample/Chr reference=1 the sample chromosome is in equimolar concentration with the reference chromosome so the patient is normal.

In the graph in step 4, there is clearly an increase in the frequency of fragments of 100 bp, compared to those of 120 bp, indicating an increase in copy number of the starting material—which in this case suggests an increase in the number of copies of Chromosome 21-indicative of Down's Syndrome.

Method Summary with Reference to FIG. 12

With reference to FIG. 12, a summary of the method is as follows.

In the figures the cross hatched areas represent Chromosome 21 and the area hatched with vertical lines represents a reference Chromosome 2. Areas with a dotted fill represent all or part of a non-palindromic restricting enzyme sequence.

Areas that have a solid black fill or a chequer board black and white fill are anchor sequences.

### Step 1—Symmetric PCR Step Using Chromosome Specific PCR Primers

Symmetric PCR is performed on a target sequence in Chromosome 21 and Chromosome 2 using chromosome specific PCR primers.

With respect to Chromosome 21, a tailed forward primer complementary to the chromosome is used which includes a region complementary to the target region to be amplified and an anchor sequence as a tail. A tailed reverse primer is also used which includes a non-palindromic restriction enzyme recognition sequence and an anchor sequence which both become added to the amplified sequence of Chromosome 21. This amplified sequence is also referred to as the first nucleic acid sequence.

With respect to Chromosome 2 a tailed reverse primer complementary to the chromosome and including a tail comprising an anchor sequence is used together with a tailed forward primer which includes a non-palindromic restriction enzyme recognition sequence and an anchor sequence, both of which become added to the amplified sequence of Chromosome 2. This amplified sequence is also referred to as the second nucleic acid sequence.

The sequence included for the restriction enzyme may be sequence recognised by the enzyme BstXI.

The primers which do not include the restriction enzyme cut site may be labelled with a fluorophore, such as FAM.

The result of each PCR reaction preferably produces a different sized amplification product. For the purposes of this example, the amplification involving Chromosome 21 produces an amplification product of 100 bp and the amplification product from Chromosome 2 is 120 bp.

This PCR reaction is performed for up to 10 cycles, more preferably up to 5 cycles

### Step 2—Symmetric PCR Step Using Anchor Specific PCR Primers

The DNA amplified in step 1 is then amplified further using anchor specific PCR primers which amplify both the Chromosome 2 and the Chromosome 21 derived sequences. The forward and reverse primers depicted in FIG. 12 are different, however in an alternative embodiment they may be the same.

By using the same primers for both the first and second nucleic acid sequences (Chromosome 21 and 2) the differences in hybridisation and PCR amplification efficiency introduced by the chromosome specific primers is eliminated.

Preferably at least 10, 15, 20, 25, 30, 35 or more PCR cycles are performed using the anchor specific primers.

### Step 3—Restriction Enzyme Digestion

The amplified first and second sequences are then cut with the restriction enzyme, in this case BstXI, which recognises the non-palindromic restriction enzyme recognition sequence. All the amplified Chromosome 21 sequences, or first nucleic acid sequences, will have an overhanging complementary end of the same sequence. Similarly, all the amplified Chromosome 2 sequences, or first nucleic acid sequences, will have an overhanging end of the same sequence. Due to the non-palindromic nature of the restriction enzyme recognition sequence and cut site the overhanging end on the amplified Chromosome 21 sequences will be different to the overhanging end on the amplified Chromosome 2 sequences. The different sequences are however complementary.

Purification of the amplified nucleic acids may be performed before and/or after cleavage with the restriction enzyme. In particular, purification may be performed after cleavage with the restriction enzyme to remove any small fragments produced by the cleavage.

### Step 4—Ligation and Subtractive Hybridisation

A ligation reaction is now performed to anneal the cleaved amplified Chromosome 21 and the cleaved amplified Chromosome 2 sequence in a 1:1 ratio.

There are now essentially just three sequences in a reaction tube: firstly unligated but amplified Chromosome 21 sequence which is 100 bp; secondly unligated but amplified Chromosome 2 sequence which is 120 bp; and finally ligated Chromosome 21 and 2 sequences which is 220 bp. The new ligated sequence can then be subtracted from Chromosome 21 and 2 sequences by using size discrimination. This may be achieved by any suitable method, for example gel electrophoresis or if a floruophore has been incorporated a fragment analyser may be used to discriminate the different fragments in the tube.

### Step 5—Determination of Fragments Present Using a Fragment Analyser

If a fragment analyser is used the ratio of the area of the peak generated by the sample chromosome (in this case Chromosome 21) and the reference chromosome (in this case Chromosome 2) sequences can be used to test normal and abnormal patients. If the ratio of Chr sample/Chr reference >1 theoretically the patient has more sample chromosome in the reaction tube suggesting an aneuploidy of the patient—in this case an increase in the number of copies of chromosome 21, hence allowing a diagnosis of Down's Syndrome. If Chr sample/Chr reference <1 theoretically the patient has less sample chromosome in the reaction tube again suggesting an aneuploidy of the patient and if Chr sample/Chr reference=1 the sample chromosome is in equimolar concentration with the reference chromosome so the patient is normal.

In the graph in step 5, there is clearly an increase in the frequency of fragments of 100 bp, compared to those of 120 bp, indicating an increase in copy number of the starting material—which in this case suggests an increase in the number of copies of Chromosome 21—indicative of Down's Syndrome.

In a further embodiment the method of this embodiment and indeed all embodiments may be applied to more than sequence, as illustrated in FIGS. 13A and 13B, which depicts a situation where more than one sequence is amplified and studied. Preferably all sequences on the sample sequence, for example the sample chromosome, such as Chromosome 21, are the same length. Preferably all sequences on the reference sequence, for example the reference chromosome, such as Chromosome 2, are the same length. Preferably the sequences on the sample chromosome are different in length to the sequences on the reference chromosome. The amplification of just two sequences from a genomic DNA template can be complicated due to the differing gc content and hybridisation efficiency of the primers used to amplify these two sequences. A way to overcome this problem is to exploit the averaging effect of using multiple sequences from the genomic DNA template belonging to a sample chromosome and a reference chromosome. This may be further improved by using primers that are engineered to have anchors to amplify all the sequences from sample and reference chromosomes. Further reducing the scope of unequal amplification. The anchor on each primer may be the same, such that the final amplification uses the same primers for amplification of both the first and second target region.

### First Specific Example of the Third Embodiment of the Invention

There now follows a more specific example of the third embodiment of the invention, detailing the material and methods used. In this example genomic DNA is first amplified with chromosome specific primers and then anchor specific primers are used.

### Materials

Peripheral blood leukocyte genomic DNA (100 μg) is obtained from BioChain (Hayward, Calif. USA) Cat No. D1234148.
FastDigest BstXI restriction enzyme 100 μl (for 100 reactions) is obtained from Fermentas (Vilnius, Lithuania) Cat No. FD1024.
Agarose, molecular biology grade reagent (500 g) is obtained from Helena Biosciences (Tyne and Wear, UK) Cat No. 8201-07.
DEPC-treated H₂O, pyrogen-free (100 ml) Cat No. 75-0024, Platinum Taq polymerase kit (5 u/μl) Cat No. 10966-034 and T4 DNA ligase (1 u/μl) Cat No 15224-017 are obtained from Invitrogen (Abingdon, Oxfordshire, UK).
QIAquick PCR purification kit (50 reactions) is obtained from QIAGEN Ltd (Crawley, West Sussex, UK) Cat No. 28104.
Ethidium bromide (10 mg/ml) is obtained from Sigma-Aldrich Ltd (Gillingham, Dorset, UK) Cat No. E1510-10 ml.
MicroCLEAN DNA Cleanup Reagent, 5×1 ml is obtained from Web Scientific Ltd (Crew, UK) Cat No 2MCL-5.

### Primers

| | Chromosome | | |
|---|---|---|---|
| | location | | |
| Primer | (NCBI | | |
| Name | 36.3) | Sequence | Details |
| Chr2-28 | 2p | 5′gga gaa agc agc | Genomic |
| F(BstXI) | | cct cca ttg | DNA |
| | | gag cta aca | |
| | | gct tct gtc tt | |
| | | 3′ | |
| Chr2-28 | 2p | 5′ ctc cta cag agg | Genomic |
| R(StuI) | | cct cca ctc tct tgg | DNA |
| | | gaa ggc tcg ggg tga | |
| | | gtc 3′ | |
| Chr21-58 | 21q | 5′ctc cta cag agg | Genomic |
| R(BstXI) | | cct cca cgt ctc tca | DNA |
| | | ctc cct gca ct 3′ | |
| Chr21-58 | 21q | 5′ gga gaa agc agg | Genomic |
| F (StuI) | | cct cca aga gag tgg | DNA |
| | | agg aaa ccc agc gag | |
| | | cag 3′ | |
| Anchor 1 | | 5′ FAM-gga gaa | Genomic |
| | | agc agg cct cca 3′ | DNA |
| Anchor 2 | | 5′ FAM-ctc cta cag | Genomic |
| | | agg cct cca 3′ | DNA |

In this example the reference chromosome is Chromosome 2. The primers would produce a 146 bp fragment from Chromosome 2 and 165 bp fragment from Chromosome 21. All primers are obtained from Applied Biosystems California, USA.

### Method

### PCR

### Specific Chromosomal Amplification PCR Protocol

| | Volume | |
|---|---|---|
| Reagent | (final concentration) | |
| Betaine (2.6M Betaine in 2.6% DMSO) | 12.5 | μl |
| 10 x PCR buffer | 2.5 | μl (1 x PCR buffer) |
| 10 mM dNTPs | 1.0 | μl (0.1 mM) |
| 50 mM MgCl₂ | 1.5 | μl (3 mM) |
| 10 μm Forward primer(Chr 21) | 0.50 | μl (0.2 μM) |
| 10 μm Reverse primer(Chr 21) | 0.50 | μl (0.2 μM) |
| 10 μm Forward primer(Chr 2) | 0.50 | μl (0.2 μM) |
| 10 μm Reverse primer(Chr 2) | 0.50 | μl (0.2 μM) |
| Taq DNA polymerase (Invitrogen) | 0.3 | μl (1.5 units) |
| Plasma DNA input | 5.2 | μl |
| Final total volume | 25.0 | μl |

A standard PCR cycle of 1 cycle (95° C. 3 min) followed by 5 cycles (94° C. 30 s, 57° C. 20 min, 72° C. 30 s) and a holding step at 4° C. is used.

After amplification by Specific Chromosomal PCR each sample is then processed by QIAquick PCR column according to the manufacturer's protocol or MicroCLEAN DNA clean up reagent. The Cleaned DNA is then eluted in 30 μl of H₂O and used in the anchor standard PCR protocol.

### Anchor Standard PCR Protocol

| | Volume | |
|---|---|---|
| Reagent | (final concentration) | |
| H₂O | 4.2 | μl |
| Betaine (2.6M Betaine in 2.6% DMSO) | 12.5 | μl |
| 10 x PCR buffer | 2.5 | μl (1 x PCR buffer) |
| 10 mM dNTPs | 1.0 | μl (0.1 mM) |
| 50 mM MgCl₂ | 1.5 | μl (3 mM) |
| 10 μm Anchor 1 | 1 | μl (0.4 μM) |
| 10 μm Anchor 2 | 1 | μl (0.4 μM) |
| Taq DNA polymerase (Invitrogen) | 0.3 | μl (1.5 units) |
| Total volume of reagents | 24.0 | μl |
| DNA input from Chromosomal amplification | 1.0 | μl |
| Final total volume | 25.0 | μl |

A standard PCR cycle used was 1 cycle (95° C. 3 min) followed by 35 cycles (94° C. 30 s, 55° C. 60 s, 72° C. 30 s) and a holding step at 4° C.

After Anchor Standard PCR each sample is then processed by QIAquick PCR column according to the manufacturer's protocol or MicroCLEAN DNA clean up reagent. The Cleaned DNA is then eluted in 30 μl of H₂O. The eluted DNA will be digested with the BstXI enzyme.

### Enzyme Restriction

BstXI restriction digestion of Chromosomal Amplification-specific PCR:

### Enzyme Mix:

Fast Digest BstXI enzyme 1 μl
10× Fast Digest BstXI buffer 4 μl

### DDH₂O 5 μl

Final reaction volume Σ40 μl

10 μl of enzyme mix is used to digest each PCR product. Samples are incubated at 37° C. for 20 minutes.

After digestion the DNA is then processed by QIAquick PCR column according to the manufacturer's protocol or MicroCLEAN DNA clean up reagent. The DNA is then eluted in 30 μl H₂O. The eluted DNA is divided into two aliquots of 15 μl The first aliquot is incubated in T4 DNA Ligase at 4° C. overnight and the second is used as a control for the ligation.

### Ligation Mix

T4 DNA Ligase enzyme 1 μl (1 u)

### 5×DNA Ligase Reaction Buffer 4 μl

Final reaction volume Σ20 μl
5 μl of Ligation mix is used to ligate each digested sample. Samples are incubated at 4° C. overnight.

The ligation samples are then analysed by a fragment analyser and the ratios of the areas of the sample chromosome and the reference chromosome will establish if the patient is normal. The fragment analyser may be Applied Biosystem 3130 Genetic Analyser.

### Second Specific Example of the Third Embodiment of the Invention

There now follows a more specific example of the third embodiment of the invention, detailing the material and methods used. In this example genomic DNA is first amplified using whole genomic amplification before chromosome specific primers are used.

The amplification of just two sequences from a genomic DNA template can be complicated due to the differing gc-content and hybridisation efficiency of the primers used to amplify these two sequences. In practice, two sequences that have the same number of molecules in a sample after conventional amplification could end with different numbers of molecules. To overcome this problem a commercially available system of whole genome amplification is used, this uses the “Whole Genome Amplification (WGA4) Kit” available form Sigma which amplifies all of the sequences of DNA in a given sample, keeping the differences due to conventional amplification to a minimum. Once whole genome has been amplified the sample DNA is now available in micrograms and can then be used in any method of the invention. In this particular example only chromosome specific primers are used.

### Materials

Peripheral blood leukocyte genomic DNA (100 μg) is obtained from BioChain (Hayward, Calif. USA) Cat No. D1234148.
FastDigest BstXI restriction enzyme 100 μl (for 100 reactions) Cat No. FD1024 and dUTP, 100 μM Solution Cat No. R0133 are obtained from Fermentas (Vilnius, Lithuania).
Agarose, molecular biology grade reagent (500 g) is obtained from Helena Biosciences (Tyne and Wear, UK) Cat No. 8201-07.
DEPC-treated H₂O, pyrogen-free (100 ml) Cat No. 75-0024, Platinum Taq polymerase kit (5 u/μl) Cat No. 10966-034 and T4 DNA ligase (1 u/μl) Cat No 15224-017 are obtained from Invitrogen (Abingdon, Oxfordshire, UK).
QIAquick PCR purification kit (50 reactions) is obtained from QIAGEN Ltd (Crawley, West Sussex, UK) Cat No. 28104.
Ethidium bromide (10 mg/ml) and WGA4 GenomePlex Single Cell Whole Genome Amplification Kit. Cat NoWGA4-50RXN are obtained from Sigma-Aldrich Ltd (Gillingham, Dorset, UK) Cat No. E1510-10 ml.
MicroCLEAN DNA Cleanup Reagent, 5×1 ml is obtained from Web Scientific Ltd (Crew, UK) Cat No 2MCL-5.
Primers—from Applied Biosystems, California, USA

| | Chromosome | | |
|---|---|---|---|
| | location | | |
| Primer | (NCBI | | |
| Name | 36.3) | Sequence | Details |
| Chr2-28 | 2p | 5′gga gaa agc cca | Genomic |
| F (FAM) | | ttg gag cta aca gct | DNA |
| | | tct gtc tt 3′ | |
| Chr2-28 | 2p | 5′ ctc cta cag cca | Genomic |
| R(BstXI) | | ctc tct tgg gaa ggc | DNA |
| | | tcg ggg tga gtc 3′ | |
| Chr21-58 | 21q | 5′ctc cta cag cca cgt | Genomic |
| R(FAM) | | ctc tca ctc cct gca ct | DNA |
| | | 3′ | |
| Chr21-58 | 21q | 5′ gga gaa agc cca | Genomic |
| F(BstXI) | | aga gag tgg agg aaa | DNA |
| | | ccc agc gag cag 3′ | |

In this example the reference chromosome is Chromosome 2. The primers would produce a 146 bp fragment from Chromosome 2 and 165 bp fragment from Chromosome 21.

### Methods

### Whole Genome Amplification

The DNA extracted from one millilitre of plasma is extracted following the protocol suggested by the WGA4 Kit Sigma Whole Genome Amplification Advisor document (Sigma-Aldrich Ltd., UK) with the following modifications. Briefly, 10 microlitres of plasma DNA is combined with 1 microlitre of 10× Single Cell Lysis & Fragmentation Buffer and incubated at 95 degrees Celsius for four minutes. Then the samples are immediately cooled on ice and spun down ready to be used for the library preparation following the WGA4 protocol. In the last two cycles of the amplification step 0.5 microlitres of dUTP (Uracil) at 100 micromolar is added to the PCR mix.

Uracil competes in the PCR with dTTP (thymidine) and will be incorporated into the double-stranded DNA instead of thymidine. This incorporation step will be very useful when using the restriction enzyme. The incorporated uracil in the DNA amplified with the WGA4 blocks the cleavage by restriction enzymes (FastDigest BstXI Fermentas UK) differentiating between DNA amplified by WGA4 and DNA amplified with chromosome specific primers. Once the DNA is amplified it is recovered and cleaned by QIAquick PCR purification kit. After purification the amplified DNA is used as template for PCR with chromosome specif primers with a minimal amount of amplificiation cycles. Then the chromosome specific DNA is recovered and processed with a restriction enzyme and ligase.

### PCR

### Specific Chromosomal Amplification PCR Protocol

| | Volume | |
|---|---|---|
| Reagent | (final concentration) | |
| Betaine (2.6M Betaine in 2.6% DMSO) | 12.5 | μl |
| 10 x PCR buffer | 2.5 | μl (1 x PCR buffer) |
| 10 mM dNTPs | 1.0 | μl (0.1 mM) |
| 50 mM MgCl₂ | 1.5 | μl (3 mM) |
| Forward primer mix(Chr sample) Chr21- | 0.50 | μl (2 μM) |
| 58 F (BstXI) | | |
| Reverse primer mix(Chr sample) Chr21- | 0.50 | μl (2 μM) |
| 58 R (FAM) | | |
| Forward primer mix(Chr reference) - | 0.50 | μl (2 μM) |
| Chr2-28 F (FAM) | | |
| Reverse primer mix (Chr reference) Chr2- | 0.50 | μl (2 μM) |
| 28 R (BstXI) | | |
| Taq DNA polymerase (Invitrogen) | 0.3 | μl (1.5 units) |
| Plasma DNA input | 5.2 | μl |
| Final total volume | 25.0 | μl |

A standard PCR cycle used was 1 cycle (95° C. 3 min) followed by 4 cycles (94° C. 30 s, 57° C. 5 min, 72° C. 1 min) and a holding step at 4° C.

After PCR each sample is then processed by QIAquick PCR column according to the manufacturer's protocol or MicroCLEAN DNA clean up reagent. The Cleaned DNA is then eluted in 30 μl of H₂O. The eluted DNA will be digested with the BstXI enzyme.

### Enzyme Restriction

BstXI restriction digestion of Chromosomal Amplification-specific PCR:

### Enzyme Mix:

Fast Digest BstXI enzyme 1 μl
10× Fast Digest BstXI buffer 4 μl

### DDH₂O 5 μl

Final reaction volume Σ40 μl

10 μl of enzyme mix is used to digest each PCR product. Samples are incubated at 37° C. for 20 minutes.

After digestion the DNA is then processed by QIAquick PCR column according to the manufacturer's protocol or MicroCLEAN DNA clean up reagent. The DNA is then eluted in 30 μl H₂O. The eluted DNA is divided into two aliquots of 15 μl The first aliquot is incubated in T4 DNA Ligase at 4° C. overnight and the second is used as a control for the ligation.

### Ligation Mix

T4 DNA Ligase enzyme 1 μl (1 u)

### 5×DNA Ligase Reaction Buffer 4 μl

Final reaction volume Σ20 μl

5 μl of Ligation mix is used to ligate each digested sample. Samples are incubated at 4° C. overnight.

The ligation samples are then analysed by a fragment analyser and the ratios of the areas of the sample chromosome and the reference chromosome will establish if the patient is normal. The presence of FAM on the chromosome specific primers allows detection of the amplification products. The fragment analyser may be Applied Biosystem 3130 Genetic Analyser.

To demonstrate that the ligation works and than an excess of one amplified fragment can be observed the following experiment was performed. Sequences of chromosomes 21 and 2 were amplified as described above and the resulting PCR products of either 146 bp for Chromosome 2 and 165 bp for Chromosome 21 were then purified and digested with BstXI. The cut fragments were then repurified, and mixed in a ratio of 2:1. 1.5:1 or 1:1 cut fragment 2 to cut fragment 21 in the absence and presence of a ligase. The results are shown in FIG. 14. FIG. 14 demonstrates that when chromosome 2 is in excess either in a 2:1 or a 1.5:1 ratio (tracks 2 and 4 respectively, and FIGS. 16 and 18 respectively) and the fragments are ligated, an excess of the fragment from chromosome 2 can be observed. The absence of any unligated fragments when the fragments are mixed in a 1:1 ratio can be seen in FIG. 14 track 6 and FIG. 20.

FIGS. 15 to 22 represent image analysis of the gel in FIG. 14. Each track has been analysed to determine the size and amount of a particular product is present. For reference, tracks 1 and 8 show a 100 bp ladder and the bands visible represent 100, 200 and 300 bp fragments. The ligated product of the fragment from Chromosome 2 and the fragment from Chromosome 21 is 311 bp, the unligated fragment from Chromosome 2 is 146 bp and the unligated fragment from Chromosome 21 is 165 bp,

1. A method for detection of a quantitative difference between the amount of a first target region of nucleic acid and a second target region of nucleic acid in a sample, comprising the steps of:
providing the sample comprising the nucleic acid;
amplifying the first and second target regions of the nucleic acid to obtain multiple copies of a first and a second sequence of nucleic acid;
associating the amplified first sequence with the amplified second sequence to form associated nucleic acid complexes which comprise the first sequence and the second sequence in a 1:1 ratio, wherein any excess of either the first sequence or the second sequence remain un-associated;
detecting any un-associated sequences, wherein detection of any un-associated sequences is indicative of a quantitative difference between the amount of the first and second target regions of nucleic acid in the sample.

2. A method for detection of an abnormality in a gene or chromosome copy number in a sample, comprising the steps of:
providing a sample comprising nucleic acid;
amplifying the first and second target regions of the nucleic acid to obtain multiple copies of a first and a second sequence of nucleic acid;
associating the amplified first sequence with the amplified second sequence to form associated nucleic acid complexes which comprise the first sequence and the second sequence in a 1:1 ratio, wherein any excess of either the first sequence or the second sequence remain un-associated;
detecting any un-associated sequences, wherein detection of any un-associated sequences is indicative of a quantitative difference between the amount of the first and second target regions of nucleic acid in the sample, and wherein the detection of a quantitative difference is indicative of an abnormality in a gene or chromosome copy number.

3. The method of claim 2 further comprising the steps of;
identifying a first target region in the gene or chromosome the copy number of which is to be studied; and
identifying a second (reference) target region in a different gene or chromosome before amplifying the first and second target regions.

4. The method of any of claims 1 to 3 wherein the amplification uses primers which introduce a restriction enzyme recognition sequence into the double stranded first and second nucleic acid sequences resulting from the amplification.

5. The method of claim 4 further comprising the step of cutting the double stranded nucleic acid sequences using a restriction enzyme that recognises the introduced restriction enzyme recognition sequence.

6. The method of claim 5 further comprising the step of annealing the cut first nucleic acid sequence to the cut second nucleic acid sequence in a 1:1 ratio, wherein any excess of the first or second nuclei acid sequence remains un-annealed.

7. The method of any of claims 4 to 6 wherein the restriction enzyme recognition site is arranged such that when cut the first nucleic acid sequence can anneal only to the second nucleic acid and not to other first nucleic acid sequences, and the second nucleic acid sequence can anneal only to the first nucleic acid and not to other second nucleic acid sequences.

8. The method according to any preceding claim further comprising the step of eliminating the associated nucleic acid complexes before detecting any un-associated sequences.

9. The method according to any preceding claim, wherein detecting any un-associated sequences comprises amplifying any un-associated sequences and detecting any amplification product.

10. The method according to any of claims 1 to 8, wherein detecting any un-associated sequences does not comprise amplifying any un-associated sequences before detecting any the presence of any un-associated sequences.

11. The method according to any preceding claim, wherein the nucleic acid is a mixture of nucleic acid from malignant and normal/non-malignant tissue.

12. The method according to any of claims 1 to 10, where the sample comprises maternal and foetal derived nucleic acid.

13. The method according to any preceding claim, wherein the first sequence comprises the sequence of the first target region, or complement thereof, and an additional sequence provided by a primer and/or the second sequence comprises the sequence of the second target region or complement thereof, and an additional sequence provided by a primer.

14. The method according to any preceding claim, wherein the first sequence of nucleic acid is amplified from the nucleic acid using a first primer pair and the second sequence of nucleic acid is amplified from the nucleic acid using a second primer pair.

15. The method according to claim 14, wherein the at least one primer of the first primer pair and/or second primer pair comprises a sequence which form all or part of a restriction enzyme recognition site.

16. The method according to claim 14 or claim 15, wherein an affinity tag is provided on one or both primers of the primer pair.

17. The method according to any preceding claim, wherein the sense strands of the first sequence and second sequence are associated and the anti-sense strands of the first and second sequences are removed prior to the association step.

18. The method according to any preceding claim, wherein the step of associating the first sequence with the second sequence to form the associated nucleic acid complex comprises ligating the first sequence to the second sequence.

19. The method according to any of claim 17 or 18, wherein a template nucleic acid is provided to aid association of the first and second sequences.

20. The method according to claim 19, wherein the template nucleic acid comprises a first portion which is capable of hybridising to the first sequence and a second portion which is capable of hybridising to the second sequence.

21. The method according to any of claims 19 to 20, wherein un-associated sequences and/or associated nucleic acid complex are immobilised prior to an elimination step via an affinity tag on the hybridised template nucleic acid.

22. The method according to any of claims 19 to 21, wherein the associated nucleic acid complex comprises a restriction enzyme recognition site.

23. The method according to any of claims 19 to 22, further comprising cutting the associated nucleic acid complex with a restriction enzyme at one or more positions within the first and/or second sequence of nucleic acid which is hybridized to the template nucleic acid, such that the first and/or second sequence of nucleic acid is truncated.

24. The method according to claim 22, wherein the cutting reduces the size of the first and/or second sequence by at least 5 base pairs.

25. The method of any of claims 1 to 18 wherein the first and second target regions are amplified by PCR using a first primer pair and a second primer pair, wherein the first primer pair comprises:
a first tailed primer comprising a complementary portion, which is substantially complementary to a sequence of at least part of the first target region, and a tail portion comprising a first association sequence which is substantially complementary to a sequence of at least a part of the second target region, and a second primer complementary to the other strand of the first target region; and wherein the second primer pair comprises:
a second tailed primer comprising a complementary portion, which is substantially complementary to a sequence of at least part of the second target region, and a tail portion comprising a second association sequence which is substantially complementary to a sequence of at least a part of the first target region, and a second primer complementary to the other strand of the second target region;

26. The method of claim 25 wherein following amplification with the first and second primer pair the resulting first and second sequence double stranded DNA products are amplified by asymmetric PCR.

27. The method of claim 26 further comprising hybridising the amplified single stranded first nucleic acid sequence and the amplified single stranded second nucleic acid sequence using the first and second association sequence to form an associated nucleic acid complex in which the first and second sequences are associated in a 1:1 ratio, and using a polymerase to form a substantially fully double stranded double stranded nucleic acid complex;

28. The method of claim 27 further comprising detecting the presence of any single stranded DNA.

29. The method of claim 28 wherein the single stranded DNA is amplified prior to detection.

30. The method of any preceding claim which comprises amplifying more than one first target sequence and/or more than one target second sequence.

31. The method according to any preceding claim, wherein the first target region is a part of a gene, operon, or chromosome which is associated with an abnormality, a disease, disability or other clinical syndrome and the second target region is part of a gene, operon, or chromosome which is used as a control/standard, which is known to be present in normal copy number, or vice versa.

32. The method according to claim 2 or any claim dependent on claim 2, wherein an abnormality in a chromosome number is an additional copy of a whole or part of a chromosome, or a missing copy of a whole or part of a chromosome; or wherein an abnormality in a gene copy number in a subject is one or more additional copies of a gene relative to the average copy number of the gene in a sample of subjects of a general population.

33. The method according to claim 32 wherein the detection of an abnormality in a gene copy number or a chromosome copy number comprises the detection of and/or diagnosis of a condition caused or associated with an additional copy number or reduced copy number of a gene or a chromosome.

34. Use of the method according to any preceding claim to determine if an individual has an increase or decrease in gene or chromosome copy number.

35. Use of the method according to any preceding claim to determine the choice of treatment for a condition, optionally, wherein the condition is cancer; and optionally, wherein the choice of treatment is a choice of chemotherapy regime and/or agent.

36. A kit comprising one or more primers suitable for carrying out the method according to any of claims 1-33, and instructions to carry out a method as defined in any of claims 1-33.

37. A kit comprising one or more primers suitable for carrying out the method according to any of claims 1-33, and instructions to carry out a method as defined in any of claims 1-33.
